SEQUENCE LISTING (6 sequences)

SEQ ID NO: 1 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 2 - protein, 1042 aa, Homo sapiens
MSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 3 - protein, 945 aa, Homo sapiens
MITNVAFLVSLRTEATSAKPPLGTGRWILMPTMSTDSRVSPLSGLMLSRVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVELEPETELLIDSGCPAPRAEAEDSFL

SEQ ID NO: 4 - RNA, 5700 nt, Homo sapiens
agcagccagcagugacugcccgcccuaucugggacccaggaucgcucugugagcaacuuggagccagagaggagaucaacaaggaggaggagagagccggccccucagcccugcugcccagcagcagccugugcucgcccugcccaacgcagacagccagacccagggcggccccucuggcggcucugcuccucccgaaggaugcuuggggagugaggcgaagcugggccgcuccucuccccuacagcagcccccuuccuccaucccucuguucuccugagccuucaggagccugcaccaguccugccuguccuucuacucagcuguuacccacucugggaccagcagucuuucugauaacugggagagggcaguaaggaggacuuccuggagggggugacuguccagagccuggaacugugcccacaccagaagccaucagcagcaaggacaccaugcggcuuccgggugcgaugccagcucuggcccucaaaggcgagcugcuguugcugucucuccuguuacuucuggaaccacagaucucucagggccuggucgucacacccccggggccagagcuuguccucaaugucuccagcaccuucguucugaccugcucggguucagcuccgguggugugggaacggaugucccaggagcccccacaggaaauggccaaggcccaggauggcaccuucuccagcgugcucacacugaccaaccucacugggcuagacacgggagaauacuuuugcacccacaaugacucccguggacuggagaccgaugagcggaaacggcucuacaucuuugugccagaucccaccgugggcuuccucccuaaugaugccgaggaacuauucaucuuucucacggaaauaacugagaucaccauuccaugccgaguaacagacccacagcugguggugacacugcacgagaagaaaggggacguugcacugccuguccccuaugaucaccaacguggcuuuucugguaucuuugaggacagaagcuacaucugcaaaaccaccauuggggacagggagguggauucugaugccuacuaugucuacagacuccaggugucauccaucaacgucucugugaacgcagugcagacugugguccgccagggugagaacaucacccucaugugcauugugaucgggaaugagguggucaacuucgaguggacauacccccgcaaagaaagugggcggcugguggagccggugacugacuuccucuuggauaugccuuaccacauccgcuccauccugcacauccccagugccgaguuagaagacucggggaccuacaccugcaaugugacggagagugugaaugaccaucaggaugaaaaggccaucaacaucaccgugguugagagcggcuacgugcggcuccugggagaggugggcacacuacaauuugcugagcugcaucggagccggacacugcagguaguguucgaggccuacccaccgcccacuguccugugguucaaagacaaccgcacccugggcgacuccagcgcuggcgaaaucgcccuguccacgcgcaacgugucggagacccgguaugugucagagcugacacugguucgcgugaagguggcagaggcuggccacuacaccaugcgggccuuccaugaggaugcugagguccagcucuccuuccagcuacagaucaaugucccuguccgagugcuggagcuaagugagagccacccugacaguggggaacagacaguccgcugucguggccggggcaugccccagccgaacaucaucuggucugccugcagagaccucaaaagguguccacgugagcugccgcccacgcugcuggggaacaguuccgaagaggagagccagcuggagacuaacgugacguacugggaggaggagcaggaguuugagguggugagcacacugcgucugcagcacguggaucggccacugucggugcgcugcacgcugcgcaacgcugugggccaggacacgcaggaggucaucguggugccacacuccuugcccuuuaaggugguggugaucucagccauccuggcccugguggugcucaccaucaucucccuuaucauccucaucaugcuuuggcagaagaagccacguuacgagauccgauggaaggugauugagucugugagcucugacggccaugaguacaucuacguggaccccaugcagcugcccuaugacuccacgugggagcugccgcgggaccagcuugugcugggacgcacccucggcucuggggccuuugggcaggugguggaggccacggcucauggccugagccauucucaggccacgaugaaaguggccgucaagaugcuuaaauccacagcccgcagcagugagaagcaagcccuuaugucggagcugaagaucaugagucaccuugggccccaccugaacguggucaaccuguugggggccugcaccaaaggaggacccaucuauaucaucacugaguacugccgcuacggagaccugguggacuaccugcaccgcaacaaacacaccuuccugcagcaccacuccgacaagcgccgcccgcccagcgcggagcucuacagcaaugcucugcccguugggcucccccugcccagccauguguccuugaccggggagagcgacgguggcuacauggacaugagcaaggacgagucgguggacuaugugcccaugcuggacaugaaaggagacgucaaauaugcagacaucgaguccuccaacuacauggccccuuacgauaacuacguucccucugccccugagaggaccugccgagcaacuuugaucaacgagucuccagugcuaagcuacauggaccucgugggcuucagcuaccagguggccaauggcauggaguuucuggccuccaagaacugcguccacagagaccuggcggcuaggaacgugcucaucugugaaggcaagcuggucaagaucugugacuuuggccuggcucgagacaucaugcgggacucgaauuacaucuccaaaggcagcaccuuuuugccuuuaaaguggauggcuccggagagcaucuucaacagccucuacaccacccugagcgacgugugguccuucgggauccugcucugggagaucuucaccuuggguggcaccccuuacccagagcugcccaugaacgagcaguucuacaaugccaucaaacgggguuaccgcauggcccagccugcccaugccuccgacgagaucuaugagaucaugcagaagugcugggaagagaaguuugagauucggccccccuucucccagcuggugcugcuucucgagagacuguugggcgaagguuacaaaaagaaguaccagcagguggaugaggaguuucugaggagugaccacccagccauccuucggucccaggcccgcuugccuggguuccauggccuccgaucuccccuggacaccagcuccguccucuauacugccgugcagcccaaugagggugacaacgacuauaucaucccccugccugaccccaaacccgagguugcugacgagggcccacuggaggguucccccagccuagccagcuccacccugaaugaagucaacaccuccucaaccaucuccugugacagcccccuggagccccaggacgaaccagagccagagccccagcuugagcuccagguggagccggagccagagcuggaacaguugccggauucggggugcccugcgccucgggcggaagcagaggauagcuuccuguagggggcuggccccuacccugcccugccugaagcucccccccugccagcacccagcaucuccuggccuggccugaccgggcuuccugucagccaggcugcccuuaucagcuguccccuucuggaagcuuucugcuccugacguguugugccccaaacccuggggcuggcuuaggaggcaagaaaacugcaggggccgugaccagcccucugccuccagggaggccaacugacucugagccaggguucccccagggaacucaguuuucccauauguaaaaugggaaaguuaggcuugaugacccagaaucuaggauucucucccuggcugacagguggggagaccgaaucccucccugggaagauucuuggaguuacugaggugguaaauuaacuuuuuucuguucagccagcuaccccucaaggaaucauagcucucuccucgcacuuuuauccacccaggagcuagggaagagacccuagccucccuggcugcuggcugagcuagggccuagccuugagcaguguugccucauccagaagaaagccagucuccucccuaugaugccagucccugcguucccuggcccgagcuggucuggggccauuaggcagccuaauuaaugcuggaggcugagccaaguacaggacacccccagccugcagcccuugcccagggcacuuggagcacacgcagccauagcaagugccugugucccuguccuucaggcccaucaguccuggggcuuuuucuuuaucacccucagucuuaauccauccaccagagucuagaaggccagacgggccccgcaucugugaugagaauguaaaugugccaguguggaguggccacgugugugugccaguauauggcccuggcucugcauuggaccugcuaugaggcuuuggaggaaucccucacccucucugggccucaguuuccccuucaaaaaaugaauaagucggacuuauuaacucugagugccuugccagcacuaacauucuagaguauuccaggugguugcacauuuguccagaugaagcaaggccauauacccuaaacuuccauccugggggucagcugggcuccugggagauuccagaucacacaucacacucuggggacucaggaaccaugccccuuccccaggcccccagcaagucucaagaacacagcugcacaggccuugacuuagagugacagccgguguccuggaaagcccccagcagcugccccagggacaugggaagaccacgggaccucuuucacuacccacgaugaccuccggggguauccugggcaaaagggacaaagagggcaaaugagaucaccuccugcagcccaccacuccagcaccugugccgaggucugcgucgaagacagaauggacagugaggacaguuaugucuuguaaaagacaagaagcuucagauggguaccccaagaaggaugugagaggugggcgcuuuggagguuugccccucacccaccagcugccccaucccugaggcagcgcuccauggggguaugguuuugucacugcccagaccuagcagugacaucucauuguccccagcccagugggcauuggaggugccaggggagucaggguuguagccaagacgcccccgcacggggaggguugggaagggggugcaggaagcucaaccccucugggcaccaacccugcauugcagguuggcaccuuacuucccugggauccccagaguugguccaaggagggagaguggguucucaauacgguaccaaagauauaaucaccuagguuuacaaauauuuuuaggacucacguuaacucacauuuauacagcagaaaugcuauuuuguaugcuguugaguuuuucuaucuguguacuuuuuuuuaagggaaagauuuuaauauuaaaccuggugcuucucacucaca

SEQ ID NO: 5 - RNA, 5654 nt, Homo sapiens
agcagccagcagugacugcccgcccuaucugggacccaggaucgcucugugagcaacuuggagccagagaggagaucaacaaggaggaggagagagccggccccucagcccugcugcccagcagcagccugugcucgcccugcccaacgcagacagccagacccagggcggccccucuggcggcucugcuccucccgaaggaugcuuggggagugaggcgaagcugggccgcuccucuccccuacagcagcccccuuccuccaucccucuguucuccugagccuucaggagccugcaccaguccugccuguccuucuacucagcuguuacccacucugggaccagcagucuuucugauaacugggagagggcaguaaggaggacuuccuggagggggugacuguccagagccuggaacugugcccacaccagaagccaucagcagcaaggcgagcugcuguugcugucucuccuguuacuucuggaaccacagaucucucagggccuggucgucacacccccggggccagagcuuguccucaaugucuccagcaccuucguucugaccugcucggguucagcuccgguggugugggaacggaugucccaggagcccccacaggaaauggccaaggcccaggauggcaccuucuccagcgugcucacacugaccaaccucacugggcuagacacgggagaauacuuuugcacccacaaugacucccguggacuggagaccgaugagcggaaacggcucuacaucuuugugccagaucccaccgugggcuuccucccuaaugaugccgaggaacuauucaucuuucucacggaaauaacugagaucaccauuccaugccgaguaacagacccacagcugguggugacacugcacgagaagaaaggggacguugcacugccuguccccuaugaucaccaacguggcuuuucugguaucuuugaggacagaagcuacaucugcaaaaccaccauuggggacagggagguggauucugaugccuacuaugucuacagacuccaggugucauccaucaacgucucugugaacgcagugcagacugugguccgccagggugagaacaucacccucaugugcauugugaucgggaaugagguggucaacuucgaguggacauacccccgcaaagaaagugggcggcugguggagccggugacugacuuccucuuggauaugccuuaccacauccgcuccauccugcacauccccagugccgaguuagaagacucggggaccuacaccugcaaugugacggagagugugaaugaccaucaggaugaaaaggccaucaacaucaccgugguugagagcggcuacgugcggcuccugggagaggugggcacacuacaauuugcugagcugcaucggagccggacacugcagguaguguucgaggccuacccaccgcccacuguccugugguucaaagacaaccgcacccugggcgacuccagcgcuggcgaaaucgcccuguccacgcgcaacgugucggagacccgguaugugucagagcugacacugguucgcgugaagguggcagaggcuggccacuacaccaugcgggccuuccaugaggaugcugagguccagcucuccuuccagcuacagaucaaugucccuguccgagugcuggagcuaagugagagccacccugacaguggggaacagacaguccgcugucguggccggggcaugccccagccgaacaucaucuggucugccugcagagaccucaaaagguguccacgugagcugccgcccacgcugcuggggaacaguuccgaagaggagagccagcuggagacuaacgugacguacugggaggaggagcaggaguuugagguggugagcacacugcgucugcagcacguggaucggccacugucggugcgcugcacgcugcgcaacgcugugggccaggacacgcaggaggucaucguggugccacacuccuugcccuuuaaggugguggugaucucagccauccuggcccugguggugcucaccaucaucucccuuaucauccucaucaugcuuuggcagaagaagccacguuacgagauccgauggaaggugauugagucugugagcucugacggccaugaguacaucuacguggaccccaugcagcugcccuaugacuccacgugggagcugccgcgggaccagcuugugcugggacgcacccucggcucuggggccuuugggcaggugguggaggccacggcucauggccugagccauucucaggccacgaugaaaguggccgucaagaugcuuaaauccacagcccgcagcagugagaagcaagcccuuaugucggagcugaagaucaugagucaccuugggccccaccugaacguggucaaccuguugggggccugcaccaaaggaggacccaucuauaucaucacugaguacugccgcuacggagaccugguggacuaccugcaccgcaacaaacacaccuuccugcagcaccacuccgacaagcgccgcccgcccagcgcggagcucuacagcaaugcucugcccguugggcucccccugcccagccauguguccuugaccggggagagcgacgguggcuacauggacaugagcaaggacgagucgguggacuaugugcccaugcuggacaugaaaggagacgucaaauaugcagacaucgaguccuccaacuacauggccccuuacgauaacuacguucccucugccccugagaggaccugccgagcaacuuugaucaacgagucuccagugcuaagcuacauggaccucgugggcuucagcuaccagguggccaauggcauggaguuucuggccuccaagaacugcguccacagagaccuggcggcuaggaacgugcucaucugugaaggcaagcuggucaagaucugugacuuuggccuggcucgagacaucaugcgggacucgaauuacaucuccaaaggcagcaccuuuuugccuuuaaaguggauggcuccggagagcaucuucaacagccucuacaccacccugagcgacgugugguccuucgggauccugcucugggagaucuucaccuuggguggcaccccuuacccagagcugcccaugaacgagcaguucuacaaugccaucaaacgggguuaccgcauggcccagccugcccaugccuccgacgagaucuaugagaucaugcagaagugcugggaagagaaguuugagauucggccccccuucucccagcuggugcugcuucucgagagacuguugggcgaagguuacaaaaagaaguaccagcagguggaugaggaguuucugaggagugaccacccagccauccuucggucccaggcccgcuugccuggguuccauggccuccgaucuccccuggacaccagcuccguccucuauacugccgugcagcccaaugagggugacaacgacuauaucaucccccugccugaccccaaacccgagguugcugacgagggcccacuggaggguucccccagccuagccagcuccacccugaaugaagucaacaccuccucaaccaucuccugugacagcccccuggagccccaggacgaaccagagccagagccccagcuugagcuccagguggagccggagccagagcuggaacaguugccggauucggggugcccugcgccucgggcggaagcagaggauagcuuccuguagggggcuggccccuacccugcccugccugaagcucccccccugccagcacccagcaucuccuggccuggccugaccgggcuuccugucagccaggcugcccuuaucagcuguccccuucuggaagcuuucugcuccugacguguugugccccaaacccuggggcuggcuuaggaggcaagaaaacugcaggggccgugaccagcccucugccuccagggaggccaacugacucugagccaggguucccccagggaacucaguuuucccauauguaaaaugggaaaguuaggcuugaugacccagaaucuaggauucucucccuggcugacagguggggagaccgaaucccucccugggaagauucuuggaguuacugaggugguaaauuaacuuuuuucuguucagccagcuaccccucaaggaaucauagcucucuccucgcacuuuuauccacccaggagcuagggaagagacccuagccucccuggcugcuggcugagcuagggccuagccuugagcaguguugccucauccagaagaaagccagucuccucccuaugaugccagucccugcguucccuggcccgagcuggucuggggccauuaggcagccuaauuaaugcuggaggcugagccaaguacaggacacccccagccugcagcccuugcccagggcacuuggagcacacgcagccauagcaagugccugugucccuguccuucaggcccaucaguccuggggcuuuuucuuuaucacccucagucuuaauccauccaccagagucuagaaggccagacgggccccgcaucugugaugagaauguaaaugugccaguguggaguggccacgugugugugccaguauauggcccuggcucugcauuggaccugcuaugaggcuuuggaggaaucccucacccucucugggccucaguuuccccuucaaaaaaugaauaagucggacuuauuaacucugagugccuugccagcacuaacauucuagaguauuccaggugguugcacauuuguccagaugaagcaaggccauauacccuaaacuuccauccugggggucagcugggcuccugggagauuccagaucacacaucacacucuggggacucaggaaccaugccccuuccccaggcccccagcaagucucaagaacacagcugcacaggccuugacuuagagugacagccgguguccuggaaagcccccagcagcugccccagggacaugggaagaccacgggaccucuuucacuacccacgaugaccuccggggguauccugggcaaaagggacaaagagggcaaaugagaucaccuccugcagcccaccacuccagcaccugugccgaggucugcgucgaagacagaauggacagugaggacaguuaugucuuguaaaagacaagaagcuucagauggguaccccaagaaggaugugagaggugggcgcuuuggagguuugccccucacccaccagcugccccaucccugaggcagcgcuccauggggguaugguuuugucacugcccagaccuagcagugacaucucauuguccccagcccagugggcauuggaggugccaggggagucaggguuguagccaagacgcccccgcacggggaggguugggaagggggugcaggaagcucaaccccucugggcaccaacccugcauugcagguuggcaccuuacuucccugggauccccagaguugguccaaggagggagaguggguucucaauacgguaccaaagauauaaucaccuagguuuacaaauauuuuuaggacucacguuaacucacauuuauacagcagaaaugcuauuuuguaugcuguugaguuuuucuaucuguguacuuuuuuuuaagggaaagauuuuaauauuaaaccuggugcuucucacucaca

SEQ ID NO: 6 - RNA, 5734 nt, Homo sapiens
agcagccagcagugacugcccgcccuaucugggacccaggaucgcucugugagcaacuuggagccagagaggagaucaacaaggaggaggagagagccggccccucagcccugcugcccagcagcagccugugcucgcccugcccaacgcagacagccagacccagggcggccccucuggcggcucugcuccucccgaaggaugcuuggggagugaggcgaagcugggccgcuccucuccccuacagcagcccccuuccuccaucccucuguucuccugagccuucaggagccugcaccaguccugccuguccuucuacucagcuguuacccacucugggaccagcagucuuucugauaacugggagagggcaguaaggaggacuuccuggagggggugacuguccagagccuggaacugugcccacaccagaagccaucagcagcaaggacaccaugcggcuuccgggugcgaugccagcucuggcccucaaaggcgagcugcuguugcugucucuccuguuacuucuggaaccacagaucucucagggccuggucgucacacccccggggccagagcuuguccucaaugucuccagcaccuucguucugaccugcucggguucagcuccgguggugugggaacggaugucccaggagcccccacaggaaauggccaaggcccaggauggcaccuucuccagcgugcucacacugaccaaccucacugggcuagacacgggagaauacuuuugcacccacaaugacucccguggacuggagaccgaugagcggaaacggcucuacaucuuugugccagaucccaccgugggcuuccucccuaaugaugccgaggaacuauucaucuuucucacggaaauaacugagaucaccauuccaugccgaguaacagacccacagcugguggugacacugcacgagaagaaaggggacguugcacugccuguccccuaugaucaccaacguggcuuuucugguaucuuugaggacagaagcuacaucugcaaaaccaccauuggggacagggagguggauucugaugccuacuaugucuacagacuccagggugagcccccuuucuggccugaugcucagcagagugucauccaucaacgucucugugaacgcagugcagacugugguccgccagggugagaacaucacccucaugugcauugugaucgggaaugagguggucaacuucgaguggacauacccccgcaaagaaagugggcggcugguggagccggugacugacuuccucuuggauaugccuuaccacauccgcuccauccugcacauccccagugccgaguuagaagacucggggaccuacaccugcaaugugacggagagugugaaugaccaucaggaugaaaaggccaucaacaucaccgugguugagagcggcuacgugcggcuccugggagaggugggcacacuacaauuugcugagcugcaucggagccggacacugcagguaguguucgaggccuacccaccgcccacuguccugugguucaaagacaaccgcacccugggcgacuccagcgcuggcgaaaucgcccuguccacgcgcaacgugucggagacccgguaugugucagagcugacacugguucgcgugaagguggcagaggcuggccacuacaccaugcgggccuuccaugaggaugcugagguccagcucuccuuccagcuacagaucaaugucccuguccgagugcuggagcuaagugagagccacccugacaguggggaacagacaguccgcugucguggccggggcaugccccagccgaacaucaucuggucugccugcagagaccucaaaagguguccacgugagcugccgcccacgcugcuggggaacaguuccgaagaggagagccagcuggagacuaacgugacguacugggaggaggagcaggaguuugagguggugagcacacugcgucugcagcacguggaucggccacugucggugcgcugcacgcugcgcaacgcugugggccaggacacgcaggaggucaucguggugccacacuccuugcccuuuaaggugguggugaucucagccauccuggcccugguggugcucaccaucaucucccuuaucauccucaucaugcuuuggcagaagaagccacguuacgagauccgauggaaggugauugagucugugagcucugacggccaugaguacaucuacguggaccccaugcagcugcccuaugacuccacgugggagcugccgcgggaccagcuugugcugggacgcacccucggcucuggggccuuugggcaggugguggaggccacggcucauggccugagccauucucaggccacgaugaaaguggccgucaagaugcuuaaauccacagcccgcagcagugagaagcaagcccuuaugucggagcugaagaucaugagucaccuugggccccaccugaacguggucaaccuguugggggccugcaccaaaggaggacccaucuauaucaucacugaguacugccgcuacggagaccugguggacuaccugcaccgcaacaaacacaccuuccugcagcaccacuccgacaagcgccgcccgcccagcgcggagcucuacagcaaugcucugcccguugggcucccccugcccagccauguguccuugaccggggagagcgacgguggcuacauggacaugagcaaggacgagucgguggacuaugugcccaugcuggacaugaaaggagacgucaaauaugcagacaucgaguccuccaacuacauggccccuuacgauaacuacguucccucugccccugagaggaccugccgagcaacuuugaucaacgagucuccagugcuaagcuacauggaccucgugggcuucagcuaccagguggccaauggcauggaguuucuggccuccaagaacugcguccacagagaccuggcggcuaggaacgugcucaucugugaaggcaagcuggucaagaucugugacuuuggccuggcucgagacaucaugcgggacucgaauuacaucuccaaaggcagcaccuuuuugccuuuaaaguggauggcuccggagagcaucuucaacagccucuacaccacccugagcgacgugugguccuucgggauccugcucugggagaucuucaccuuggguggcaccccuuacccagagcugcccaugaacgagcaguucuacaaugccaucaaacgggguuaccgcauggcccagccugcccaugccuccgacgagaucuaugagaucaugcagaagugcugggaagagaaguuugagauucggccccccuucucccagcuggugcugcuucucgagagacuguugggcgaagguuacaaaaagaaguaccagcagguggaugaggaguuucugaggagugaccacccagccauccuucggucccaggcccgcuugccuggguuccauggccuccgaucuccccuggacaccagcuccguccucuauacugccgugcagcccaaugagggugacaacgacuauaucaucccccugccugaccccaaacccgagguugcugacgagggcccacuggaggguucccccagccuagccagcuccacccugaaugaagucaacaccuccucaaccaucuccugugacagcccccuggagccccaggacgaaccagagccagagccccagcuugagcuccagguggagccggagccagagcuggaacaguugccggauucggggugcccugcgccucgggcggaagcagaggauagcuuccuguagggggcuggccccuacccugcccugccugaagcucccccccugccagcacccagcaucuccuggccuggccugaccgggcuuccugucagccaggcugcccuuaucagcuguccccuucuggaagcuuucugcuccugacguguugugccccaaacccuggggcuggcuuaggaggcaagaaaacugcaggggccgugaccagcccucugccuccagggaggccaacugacucugagccaggguucccccagggaacucaguuuucccauauguaaaaugggaaaguuaggcuugaugacccagaaucuaggauucucucccuggcugacagguggggagaccgaaucccucccugggaagauucuuggaguuacugaggugguaaauuaacuuuuuucuguucagccagcuaccccucaaggaaucauagcucucuccucgcacuuuuauccacccaggagcuagggaagagacccuagccucccuggcugcuggcugagcuagggccuagccuugagcaguguugccucauccagaagaaagccagucuccucccuaugaugccagucccugcguucccuggcccgagcuggucuggggccauuaggcagccuaauuaaugcuggaggcugagccaaguacaggacacccccagccugcagcccuugcccagggcacuuggagcacacgcagccauagcaagugccugugucccuguccuucaggcccaucaguccuggggcuuuuucuuuaucacccucagucuuaauccauccaccagagucuagaaggccagacgggccccgcaucugugaugagaauguaaaugugccaguguggaguggccacgugugugugccaguauauggcccuggcucugcauuggaccugcuaugaggcuuuggaggaaucccucacccucucugggccucaguuuccccuucaaaaaaugaauaagucggacuuauuaacucugagugccuugccagcacuaacauucuagaguauuccaggugguugcacauuuguccagaugaagcaaggccauauacccuaaacuuccauccugggggucagcugggcuccugggagauuccagaucacacaucacacucuggggacucaggaaccaugccccuuccccaggcccccagcaagucucaagaacacagcugcacaggccuugacuuagagugacagccgguguccuggaaagcccccagcagcugccccagggacaugggaagaccacgggaccucuuucacuacccacgaugaccuccggggguauccugggcaaaagggacaaagagggcaaaugagaucaccuccugcagcccaccacuccagcaccugugccgaggucugcgucgaagacagaauggacagugaggacaguuaugucuuguaaaagacaagaagcuucagauggguaccccaagaaggaugugagaggugggcgcuuuggagguuugccccucacccaccagcugccccaucccugaggcagcgcuccauggggguaugguuuugucacugcccagaccuagcagugacaucucauuguccccagcccagugggcauuggaggugccaggggagucaggguuguagccaagacgcccccgcacggggaggguugggaagggggugcaggaagcucaaccccucugggcaccaacccugcauugcagguuggcaccuuacuucccugggauccccagaguugguccaaggagggagaguggguucucaauacgguaccaaagauauaaucaccuagguuuacaaauauuuuuaggacucacguuaacucacauuuauacagcagaaaugcuauuuuguaugcuguugaguuuuucuaucuguguacuuuuuuuuaagggaaagauuuuaauauuaaaccuggugcuucucacucaca

(19) United States
(12) Patent Application Publication
(10) Pub. No.: US 2023/0392209 A1
(43) Pub. Date: Dec. 7, 2023

(54) METHODS FOR TREATING A SUBJECT WITH LOCAL INVASIVE BREAST CANCER BASED ON PDGFRB LEVELS

(71) Applicants: Prelude Corporation, Laguna Hills, CA (US); University of Bergen, Bergen (NO); TECKNET AB, Knivsta (SE); Per-Olof Malmström, Knivsta (SE); Lars Mårten Fernö, Knivsta (SE)

(72) Inventors: Carina Strell, Stockholm (SE); Carl Arne Östman, Knivsta (SE); Axel Emanuel Stenmark Tullberg, Göteborg (SE); Erik Carl Viktor Holmberg, Göteborg (SE); Per Oswald Karlsson, Göteborg (SE); Per-Olof Malmström, Skanör (SE); Lars Mårten Fernö, Limhamn (SE); Lars Andreas Akslen, Soreidgrend (NO); Troy M. Bremer, Irvine, CA (US); Reidunn Jetne Edelmann, Bergen (NO)

(21) Appl. No.: 18/033,054

(22) PCT Filed: Oct. 20, 2021
(86) PCT No.: PCT/US2021/055810

§ 371 Date: Apr. 20, 2023

Related U.S. Application Data
(60) Provisional application No. 63/094,574, filed on Oct. 21, 2020.

(51) Int. Cl. C12Q 1/6886; G01N 33/574

(52) U.S. Cl. CPC C12Q 1/6886; G01N 33/57415; G01N 2333/49; C12Q 2600/158; G01N 2800/52; C12Q 2600/106

(57) ABSTRACT

Provided herein are various methods for treating a subject for local and/or regional recurrence of invasive breast cancer or a subject with invasive breast cancer, identifying a subject who will not be adequately responsive to radiation therapy, recommending a treatment to a subject, preventing an invasive breast cancer recurrence in a subject, preventing a local and/or regional recurrence of an invasive breast cancer in a subject, or modifying a treatment for a subject based on PDGFRb levels. In some embodiments, the methods are based on PDGFRb levels.

### CROSS-REFERENCE TO RELATED APPLICATIONS

This application claims the benefit of U.S. Provisional Application No. 63/094,574, filed on Oct. 21, 2020. The entirety of this related application is incorporated herein by reference.

### REFERENCE TO SEQUENCE LISTING

The present application is being filed along with a Sequence Listing in electronic format. The Sequence Listing is provided as a file entitled PRLUD009WO_SEQLIST.TXT, created Oct. 19, 2021, which is 49,606 bytes in size. The information in the electronic format of the Sequence Listing is incorporated herein by reference in its entirety.

### BACKGROUND

Over the last several decades, there have been various biomarkers discovered that allow practitioners to predict the risk of the onset or recurrence of a cancer in a subject.

### SUMMARY

In some embodiments, a method for treating a subject for local and/or regional recurrence of invasive breast cancer is provided. The method comprises: providing a local cancer tissue sample from a subject who has invasive breast cancer; analyzing the local cancer tissue sample for a level of stromal and/or epithelial PDGFRb; treating the subject with radiotherapy if the local cancer tissue sample has a low level of PDGFRb; and treating the subject with an alternative to standard radiotherapy if the local cancer tissue sample has a high level of PDGFRb.

In some embodiments, a method of treating a subject is provided. The method comprises: identifying a subject with invasive breast cancer that has a high level of PDGFRb; and administering an aggressive breast cancer therapy to the subject locally to where there is the high level of PDGFRb, wherein the aggressive breast cancer therapy is at least more than standard radiation.

In some embodiments, a method of identifying a subject who will not be adequately responsive to radiation therapy is provided. The method comprises: identifying a subject with invasive breast cancer; and determining if a local cancer tissue sample from the subject has a high level of PDGFRb. If the local cancer tissue sample from the subject has a high level of PDGFRb, administering an aggressive therapy to the subject. The aggressive therapy is not standard radiation therapy, and the aggressive therapy is at least: a) standard radiation with the addition of mastectomy or chemotherapy, or b) mastectomy or c) mastectomy and chemotherapy.

In some embodiments, a method for recommending a treatment to a subject is provided. The method comprises: analyzing a local cancer tissue sample for a level of PDGFRb from a subject; recommending that one treats the subject with standard radiotherapy if the local cancer tissue sample has a low level of PDGFRb; and recommending that one treats the subject with an alternative to standard radiotherapy if the local cancer tissue sample has a high level of PDGFRb.

In some embodiments, a method for preventing an invasive breast cancer recurrence in a subject is provided. The method comprises providing a cancer tissue sample from a subject who has invasive breast cancer; analyzing the cancer tissue sample for a level of PDGFRb; administering standard radiotherapy if the local cancer tissue sample has a low level of PDGFRb; and administering an alternative to standard radiotherapy if the local cancer tissue sample has a high level of PDGFRb.

In some embodiments, a method for preventing a local and/or regional recurrence of an invasive breast cancer in a subject is provided. The method comprises: receiving standard radiotherapy if a local cancer has a low level of PDGFRb; or receiving an alternative to standard radiotherapy if the invasive breast cancer has a high level of PDGFRb.

In some embodiments, a method of modifying a treatment for a subject is provided. The method comprises: identifying a subject with invasive breast cancer that has a high level of PDGFRb; and administering a breast cancer therapy to the subject, wherein the breast cancer therapy is more aggressive than a traditional breast cancer therapy, wherein the traditional breast cancer therapy is one recommended for the subject, based on the subject's risk factors excluding PDGFRb levels. Optionally, the traditional breast cancer therapy is defined by the NCCN guidelines as of October 2020. Optionally, the traditional breast cancer therapy is defined by the NCCN guidelines as of October 2021.

In some embodiments, a method of treating a subject is provided. The method comprises: identifying an incremental risk to a subject of a local recurrence of an invasive breast cancer based on a level of PDGFRb in a sample of an invasive breast cancer in the subject; and administering an aggressive breast cancer therapy to the subject based upon the incremental risk. A higher incremental risk will increase:
a) a likelihood of an aggressive breast cancer therapy that is at least more than what would be recommended by the NCCN;
b) the aggressiveness of the aggressive breast cancer; or
c) both a) and b).

In some embodiments, low level of PDGFRb denotes a level of protein expression. In some embodiments, low level of PDGFRb denotes a level of mRNA present in the sample. In some embodiments, high level of PDGFRb denotes a level of protein expression.

In some embodiments, a high or low level of PDGFRb is determined as a value relative to a control. Optionally, the control comprises a positive, a negative, or a positive and a negative control. In some embodiments, the control is an internal control or an external control or both. In some embodiments, the control includes a level defined to one or more housekeeping genes.

In some embodiments, high or low levels of PDGFRb are defined by a comparison of PDGFRb levels from local tissue sample to a control sample in a healthy subject. In some embodiments, high or low levels of PDGFRb are defined by a comparison of PDGFRb levels from the local tissue sample to a control sample from a tissue in the subject that does not include invasive cancer. In some embodiments, high or low level of PDGFRb are defined by a comparison to a standardized level set by a level of expression of a house keeping gene.

In some embodiments, treating the subject with standard radiotherapy denotes a therapy in line with the guidelines in the NCCN guidelines. Optionally, the NCCN guidelines are as of 2020. Optionally, the NCCN guidelines are as of 2021.

In some embodiments, treating the subject with an alternative to standard radiotherapy denotes either a) administering a more intense level of therapy than that outlined in the NCCN guidelines, or b) radiation boost with higher dose levels or with broader indications than in current NCCN guidelines, mastectomy, concurrent radiochemotherapy. In some embodiments, treating the subject with an alternative to standard radiotherapy denotes applying radiotherapy at an intensity of more than: 25 fractions of 2 Gy each (total 50 Gy), 15 fractions of 2.67 Gy each (total 40 Gy), 16 fractions of 2.66 Gy each (total 42.5 Gy) or 5 fractions of 5.2 Gy each (total 26 Gy). In some embodiments, treating the subject with an alternative to standard radiotherapy denotes applying radiotherapy in combination with at least one of the following: chemotherapy, endocrine therapy, anti-HER2 therapy, immunotherapy, PARP inhibitor therapy, or other targeted therapies such as tyrosine kinase inhibitors or monoclonal antibodies against PDGFRb.

In some embodiments, high PDGFRb denotes the subject who has PDGFRb levels at the highest quartile of a population of PDGFRb levels of a population of people from the SweBCG91RT clinical trial of 1991-97. In some embodiments, a high or low PDGFRb level is determined by a combination of a) intensity of staining and b) percent of positive fraction of the tumor stained, wherein greater amounts in either a) or b) result in an increased PDGFRb level. Optionally, a) is defined into four parts, and wherein b) is defined into five parts.

In some embodiments, high PDGFRb denotes the subject has PDGFRb levels at the highest 10% of a population of PDGFRb levels of a population of people. In some embodiments, high or low PDGFRb is determined as a function of staining of the fraction of whole stroma that is positive and a level of expression. In some embodiments, high or low PDGFRb is determined as a function of staining of the fraction of epithelia (e.g., tumor core tissue) and/or stroma that is positive and a level of expression in the analyzed tissue. Optionally, fraction of staining is multiplied by the level of expression.

In some embodiments, a level of PDGFRb is analyzed as a continuous metric so that a continuous risk assessment is further provided to the subject.

In some embodiments, high or low PDGFRb is determined by measuring the stromal PDGFRb staining intensity in the area of the tumor-associated stroma displaying highest PDGFRb expression. In some embodiments, high or low PDGFRb is determined by measuring the epithelial (e.g., tumor core tissue) and/or stromal PDGFRb staining intensity in the area of the tumor-associated stroma displaying highest PDGFRb expression.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: The scoring of stromal PDGFRb staining by immunohistochemistry (IHC). Staining of PDGFRb was performed on tissue microarrays and evaluated by two independent raters for average intensity. Average intensity follows a four-grade scale (0/negative; 1/low; 2/moderate; 3/high); positive stroma fraction as well as overall stroma abundance following a five-grade scale (0/0%; 1/1-10%; 2/11-50; 3/51-75%; 4/76-100%).

FIGS. 2A-2C Prognostic effect: Univariable analysis of cumulative incidence of ipsilateral breast tumor recurrence (IBTR; FIG. 2A), any recurrence (allrec; FIG. 2B) and breast cancer specific death (BCSD; FIG. 2C) in patients of different PDGFRb score groups. Red lines represent the PDGFRb low, blue the moderate and orange the high score group.

FIGS. 3A-3C Predictive effect: Univariable analysis of cumulative incidence of ipsilateral breast tumor recurrence (IBTR; FIG. 3A), any recurrence (allrec; FIG. 3B) and breast cancer specific death (BCSD; FIG. 3C) with or without adjuvant radiotherapy (RT) in patients of different PDGFRb score groups. Red lines represent patients not receiving adjuvant RT treatment (no RT) and blue lines represent adjuvant RT treated patients.

FIG. 4 depicts the combined low and moderate PDGFRb score group from the final example presented herein, and shows the radiotherapy response-predictive potential of stromal PDGFRb expression. Univariable analysis of cumulative incidence of ipsilateral breast tumor recurrence (IBTR, top panels), any recurrence (allrec, middle panels) and breast cancer specific death (BCSD, bottom panels) with or without adjuvant radiotherapy (RT) in patients of different PDGFRb score groups. Red lines represent patients not receiving adjuvant RT treatment (no RT) and blue lines represent adjuvant RT treated patients. Tables indicate numbers of patients at risk. p values are based on the cumulative incidence function (CIF) numbers over ten years since breast conserving surgery. Hazard ratios (HR) are provided for 5, 10 and 15 year time points

FIG. 5 depicts the correlation between PDGFRb score and clinicopathological parameters. Spearman's Rank test-based correlation analysis between clinicopathologic parameters and stromal PDGFRb status in patients of the SweBCG91RT trial. PDGFRb score, age, tumor size and overall stroma fraction are included as continuous variables. Histological grade comprises grade I, II and III. Estrogen receptor (ER) status is classified as yes or no. Subtype refers to subtypes Luminal A-like, Luminal B-like, HER2 positive or triple negative. Numbers indicate Spearman's rho (p); *p<0.05; **p<0.01; ***p<0.001; ****p<0.0001.

FIG. 6 depicts a CONSORT flowchart. Patients from the Swedish Breast Cancer Group 91 Radiotherapy (SweBCG91RT) randomized radiotherapy trial included in the present biomarker study. RT radiotherapy, TMA tissue microarray, PDGFRb platelet derived growth factor receptor beta.

FIG. 7 depicts Table 3, showing distribution of clinicopathological variables in the SweBCG91RT cohort depending on PDGFRb score.

FIG. 8 depicts Table 4, showing the prognostic performance of PDGFRb score group in uni- and multivariable Cox regression analysis.

FIG. 9 depicts Table 5, showing the interaction between PDGFRb score and RT treatment in Cox regression analysis.

FIGS. 10A, 10B, and 10C depict non-limiting examples of amino acid sequences of human PDGFRb protein.

FIGS. 11A, 11B, and 11C depict non-limiting examples of nucleic acid sequences of human PDGFRb mRNA.

### DETAILED DESCRIPTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Radiotherapy is a common therapy for patients with invasive breast cancer. However, radiation not only kills or slows the growth of cancer cells, it can also affect nearby healthy cells. Damage to healthy cells can cause side effects. In addition, radiotherapy can be expensive. It uses complex machines and involves the services of many health care providers. Moreover, it may not be effective to certain patients, and/or patients can develop recurrence post treatment. Some embodiments described herein use PDGFRb as a predictive marker to determine whether radiotherapy should be used for (on) patients with invasive breast cancer (and if used, how much, what form, etc.). In some situations, standard radiation can be used as part of a treatment plan along with breast surgery. In some situations, the breast surgery is breast conserving surgery, and in other cases it is mastectomy, and in others it is oncoplastic surgery. In other situations, standard radiation can be used as part of a treatment plan with surgery and chemotherapy. In other situations, the treatment plan consists of mastectomy without radiation therapy or chemotherapy. In other situations, the treatment plan consists of mastectomy and chemotherapy without radiation therapy. And in some situations, the treatment plan comprises or consists of breast conserving surgery and chemotherapy without radiation therapy. In some embodiments, the use can be use in providing or recommending a treatment plan for invasive breast cancer that is currently present. In some embodiments, the use can be an administration of a particular therapy that would not have otherwise (in the absence of the PDGFRb information) have been pursued.

Thus, the drawbacks of radiotherapy described above can be avoided for certain patients.

Provided herein are various methods for treating (including preventing) a subject from having a local and/or regional recurrence of invasive breast cancer. Generally, this can be applied to a subject who has invasive breast cancer, taking a sample of the cancer and assaying it for a level of PDGFRb within the sample. One can then treat the subject with either standard radiotherapy if the local cancer tissue sample has a low level of PDGFRb, or treat the subject with an alternative to standard radiotherapy if the local cancer tissue sample has a high level of PDGFRb. This can include aspects such as an increase in the total dose of radiotherapy, different fractionation of radiotherapy or other radiotherapy modality, e.g., protons instead of photons.

### Definitions

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

Unless explained otherwise, all technical and scientific terms used herein have the same meaning as commonly understood to one of ordinary skill in the art to which this disclosure belongs. All HUGO Gene Nomenclature Committee (HGNC) identifiers (IDs) mentioned herein are incorporated by reference in their entirety. Although methods and materials similar or equivalent to those described herein can be used in the practice or testing of the present disclosure, suitable methods and materials are described below. The materials, methods, and examples are illustrative only and not intended to be limiting.

The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject. In some embodiments, the terms “cancer” and “tumor” are used interchangeably.

“Radiotherapy” (also called radiation therapy) is a cancer treatment that uses high doses of radiation to kill cancer cells and shrink tumors and/or completely eradicate tumors.

“Stroma” is the part of a tissue or organ with a structural or connective role. It is made up of all the parts without specific functions of the organ—for example, connective tissue, blood vessels, ducts, etc. There are multiple ways of classifying tissues: one classification scheme is based on tissue functions and another analyzes their cellular components. Stromal tissue falls into the “functional” class that contributes to the body's support and movement. The cells which make up stroma tissues serve as a matrix in which the other cells are embedded. Stroma is made of various types of stromal cells.

“Epithelia” as used herein has its ordinary and customary meaning as understood by one of ordinary skill in the art, in view of the present disclosure. Epithelia of breast tissue include tissue and cells that line the lobules and terminal ducts. In some embodiments, epithelial tissue of a breast tumor includes tumor core tissue.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

“Antibody” denotes a polypeptide including at least a light chain or heavy chain immunoglobulin variable region which specifically recognizes and binds an epitope of an antigen. In some embodiments, antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. The term antibody includes intact immunoglobulins, as well the variants and portions thereof, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3. sup.rd Ed., W.H. Freeman & Co., New York, 1997.

In some embodiments, each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.”

References to “V_{H}” or “V_{H}” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “polyclonal antibody” is an antibody that is derived from different B-cell lines. Polyclonal antibodies are a mixture of immunoglobulin molecules secreted against a specific antigen, each recognizing a different epitope. These antibodies are produced by methods known to those of skill in the art, for instance, by injection of an antigen into a suitable mammal (such as a mouse, rabbit or goat) that induces the B-lymphocytes to produce IgG immunoglobulins specific for the antigen, which are then purified from the mammal's serum.

A “chimeric antibody” has framework residues from one species, such as human, and CDRs (which generally confer antigen binding) from another species, such as a murine antibody.

The term “array” denotes an arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules makes it possible to carry out a very large number of analyses on a sample at one time. In some embodiments, arrays of one or more molecule (such as an oligonucleotide probe) will occur on the array a plurality of times (such as twice), for instance to provide internal controls. The number of addressable locations on the array can vary, for example from at least one, to at least 2, to at least 5, to at least 10, at least 20, at least at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect the markers noted herein.

In some embodiments, within an array, each arrayed sample can be addressable, in that its location can be reliably and consistently determined within at least two dimensions of the array. Addressable arrays can be computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including nucleic acids to which proteins are bound, or vice versa. In some examples, an array contains antibodies to markers provided herein.

As used herein, the term “gene” means nucleic acid in the genome of a subject capable of being expressed to produce a mRNA and/or protein in addition to intervening intronic sequences and in addition to regulatory regions that control the expression of the gene, e.g., a promoter or fragment thereof.

As used herein, the term “diagnosis”, and variants thereof, such as, but not limited to “diagnose” or “diagnosing” shall include, but not be limited to, a primary diagnosis of a clinical state or any primary diagnosis of a clinical state. A diagnostic assay described herein is also useful for assessing the remission of a subject, or monitoring disease recurrence, or tumor recurrence, such as following surgery, radiation therapy, adjuvant therapy or chemotherapy, or determining the appearance of metastases of a primary tumor.

In some embodiments, a prognostic assay described herein is useful for assessing likelihood of treatment benefit, disease recurrence, tumor recurrence, or metastasis of a primary tumor, such as following surgery, radiation therapy, adjuvant therapy or chemotherapy. All such uses of the assays described herein are encompassed by the present disclosure. In some embodiments, the test can be used to predict if the patient will have an occurrence.

The term “breast tumor” denotes a neoplastic condition of breast tissue that can be benign or malignant. The term “tumor” is synonymous with “neoplasm” and “lesion”. Exemplary breast tumors include invasive breast cancer, DCIS, lobular carcinoma in situ (LCIS), and atypical ductal hyperplasia (ADH).

The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject, such as, but not limited to, invasive breast cancer.

The term “intraductal lesion” refers to tumors that are confined to the interior of the mammary ducts and are, therefore, not invasive breast cancers. Exemplary intraductal lesions include ADH and DCIS.

ADH is a neoplastic intraductal (non-invasive) lesion characterized by proliferation of evenly distributed, monomorphic mammary epithelial cells.

DCIS is a neoplastic intraductal (non-invasive) lesion characterized by increased mammary epithelial proliferation with subtle to marked cellular atypia. DCIS has been divided into grades (low, intermediate, and high) based on factors such as nuclear atypia, intraluminal necrosis, mitotic activity etc. Low-grade DCIS and ADH are morphologically identical, and ADH is distinguished from DCIS based on the extent of the lesion, as determined by its size and/or the number of involved ducts. DCIS is initially typically diagnosed from a tissue biopsy triggered by a suspicious finding (e.g., microcalcifications, unusual mass, tissue distortion or asymmetry, etc.) on a mammogram and/or ultrasound imaging test. It may be from routine screening imaging or, more rarely, from diagnostic imaging triggered by a positive physical examination (e.g., a palpable mass, nipple discharge, skin change, etc.) or by a significant change in a previously identified mass.

Cellular proliferation in DCIS is confined to the milk ducts. If the proliferating cells have invaded through the basement membrane of the myoepithelial cell (MEC) layer lining the duct, thus appearing in the surrounding stroma, then the lesion is considered an invasive breast cancer, even if DCIS is also present. In some cases, the invasion is very minimal (microinvasion) or the only evidence of invasion is disruption of the MEC layer (e.g., by observing discontinuities in MEC-specific protein marker stains such as SMMHC and/or p63). Typically, these microinvasive cases are treated as invasive breast cancers, although there is some controversy in the treatment of these cases.

Recurrence rates in DCIS with current treatments are difficult to estimate. However, it is likely that about 20% of patients treated with lumpectomy (who receive lumpectomies) without any further treatment would experience recurrence events within 10 years, approximately evenly split between DCIS and invasive events, while <2% of patients treated with mastectomy (who receive mastectomies) would experience recurrence. Standard of care after (with) lumpectomy is to receive adjuvant radiotherapy or adjuvant radiation (radiation therapy) (RT). Several randomized clinical trials provide evidence that adjuvant radiation therapy following lumpectomy reduces recurrence risk by approximately half for both DCIS and invasive event types, and that current clinical and pathologic assessment techniques cannot identify a low-risk sub-group in which there is no benefit from radiation therapy. Radiation is not typically administered after mastectomy. Importantly, although radiation reduces the risk of recurrence events, a survival benefit has not been established with radiation like it has for invasive breast cancer.

LCIS is non-invasive lesion that originates in mammary terminal duct-lobular units generally composed of small and often loosely cohesive cells. When it has spread into the ducts, it can be differentiated from DCIS based on morphology and/or marker stains.

The term “invasive breast cancer” denotes a malignant tumor distinct from, and non-overlapping with, ADH and DCIS, in which the tumor cells have invaded adjacent tissue outside of the mammary duct structures. It can be divided into stages (I, IIA, IIB, IIIA, IIIB, and IV).

Surgery is a treatment for a breast tumor and is frequently involved in diagnosis. The type of surgery depends upon how widespread the tumor is when diagnosed (the tumor stage), as well as the type and grade of tumor. The term “treatment” as provided herein does not require the complete or 100% curing of the subject. Instead, it encompasses the broader concept or delaying the onset of one or more symptoms, extending the life and/or quality of life of the subject, reducing the severity of one or more symptoms, etc.

“Risk” herein is the likelihood for a subject diagnosed with invasive breast cancer to have a subsequent ipsilateral breast event after having a first invasive breast cancer event. “Risk of invasive breast cancer”, denotes a risk of developing (or being diagnosed with) a subsequent invasive breast cancer in the same (a.k.a. ipsilateral) breast.

In some embodiments, surgery can include a lumpectomy, multiple lumpectomies, mastectomy, and/or bilateral mastectomy.

Adjuvant chemotherapy is often used after surgery to treat any residual disease. Systemic chemotherapy often includes a platinum derivative with a taxane. Adjuvant chemotherapy is also used to treat subjects who have a recurrence or metastasis.

“Adjuvant DCIS treatment” denotes any treatment that is appropriate for a subject that is likely to have a subsequent DCIS event, which can include, less aggressive to more aggressive treatment options depending on the risk profile and perceived patient benefit, from frequent monitoring with planned subsequent lumpectomy upon early detection of a breast event, to lumpectomy without radiation, to an additional lumpectomy, to wide excision. In some embodiments, a subject at risk of DCIS recurrence, but not invasive breast cancer can receive adjuvant DCIS treatment (optionally, in combination with any of the embodiments provided herein).

“Adjuvant invasive breast cancer treatment” denotes any treatment that is appropriate for a subject that is likely to have an invasive breast cancer occurrence, which can include, lumpectomy with radiation, to lumpectomy with targeted therapy and/or chemotherapy, to lumpectomy with radiation with targeted therapy and/or chemotherapy, to mastectomy, to mastectomy with targeted therapy and/or chemotherapy, to mastectomy with radiation, to mastectomy with radiation and targeted therapy and/or chemotherapy, to surgery with a chemotherapy In some embodiments, the targeted therapy can include: endocrine therapy (blocking hormone receptors); anti-HER2 therapy (blocking the HER2 receptor which is overexpressed in some breast cancers); immunotherapy (blocking molecules which inhibit the immune response); conjugated monoclonal antibodies (an antibody conjugated to chemotherapy (the antibody will function as a homing device to bring the chemotherapy specifically to the cancer cells); tyrosine kinase inhibitors (blocks PDGFR signaling and other receptors relying on tyrosine kinase signaling); anti-PDGFRb antibodies (blocks PDGFRb); PARP inhibitor therapy (blocking poly ADP ribose polymerase (PARP) activity), etc.

In some embodiments, chemotherapy can also be used to treat patients who have locoregional recurrences.

A “marker” refers to a measured biological component such as a protein, mRNA transcript, or a level of DNA amplification. The risk of a subsequent ipsilateral breast event can be predicted through various sets or markers that in combination allow for the prediction of whether or not a subject who has invasive breast cancer is likely to experience a subsequent ipsilateral invasive breast cancer.

The term “control” refers to a sample or standard used for comparison with a sample which is being examined, processed, characterized, analyzed, etc. In some embodiments, the control is a sample obtained from a healthy patient or a non-tumor tissue sample obtained from a patient diagnosed with a breast tumor. In some embodiments, the control is a historical control or standard reference value or range of values (such as a previously tested control sample, such as a group of breast tumor patients with poor prognosis, or group of samples that represent baseline or normal values, such as the level of cancer-associated genes in non-tumor tissue).

The term “non-radiation therapy” denotes a therapy that is adequate for addressing or reducing the risk of invasive breast cancer in a subject, and that does not derive its therapeutic effect by radiation. Examples of such therapy include, without limitation, chemo therapeutics, targeted and non-targeted, immune and non-immune modulated, monoclonal, other targeted and non-targeted, genomic therapies, antibody therapeutics, including, HER2 antibodies, including Trastuzumab. Non-radiation therapy can include, without limitation, a PARP inhibitor, including Olaparib or Talazoparib. Often, in the present application, “non-radiation therapy” is denoted as “other therapy”.

As used herein, “aggressive therapy” denotes a therapy that is more aggressive than standard therapy, for example, standard radiation therapy. In some contexts, this is defined in comparison to other therapies. In some contexts, this is defined against the current standard of care, such as the NCCN guidelines, as of October 2020. In the later situation, a subject can receive an aggressive therapy when the PDGFRb signal demands, and when standard radiation therapy would not have been predicted to be successful, based on this marker. In the former situation, an aggressive therapy can be, for example (but not limited to) a therapy that is not standard radiation therapy, and is at least: a) standard radiation with the addition of radiation boost (sequential or simultaneously integrated) or brachytherapy, mastectomy or chemotherapy, or b) mastectomy or c) mastectomy and chemotherapy. An aggressive therapy can be, for example, a therapy that is not standard radiation therapy, but has an increased total dose of radiation above the dose defined by a standard radiation therapy. In some embodiments, the therapy can include a systemic therapy, radiosensitizers, immunotherapy, or other targeted therapies. In some embodiments, therapies can include targeted therapies such as anti-HER2 therapy, immunotherapy, PARP inhibitor therapy, and/or endocrine therapy. In some embodiments, the standard therapy is defined per the NCCN Guidelines® (NCCN Clinical Practice Guidelines in Oncology) Version 8.2021, Sep. 13, 2021.

“Subject” and “patient” are used interchangeably herein, and generally refer to a human subject or patient. In some embodiments, the subject is a female. In some embodiments, the subject is a male. In some embodiments, the subject is an adult subject. In some embodiments, the subject is 18, 20, 25, 30, 35, 40, 45, 50, 55, 60, 65, 70, 75, or 80 years old or older, or an age in a range defined by any two of the preceding values. In some embodiments the subject is 18-30, 30-40, 40-50, 50-60, 60-70, or 70-80 years old.

“Recurrence” as used herein refers to a cancer that comes back, e.g., after treatment to remove or eliminate the initial cancer. “Local recurrence” refers to a cancer that comes back in the same place it first started. “Regional recurrence” refers to a cancer that comes back in the lymph nodes near the place it first started. Recurrence can be, without limitation, within 1, 2, 3, 4, 5, 6, 7, 8, 9, 10, 11, 12, 13, 14, 15, 16, 17, 18, 19, 20 years or more from an earlier treatment to remove the cancer.

### Various Embodiments

Invasive breast cancer occurs when cancer cells from inside the milk ducts or lobules break out into nearby breast tissue. Cancer cells can travel from the breast to other parts of the body through the blood stream or the lymphatic system. They may travel early in the process when a tumor is small or later when a tumor is large. “Local cancer tissues” refer to tissues where the cancer began. In some embodiments, the local cancer tissue sample is from a subject who has early state invasive breast cancer.

Platelet-derived growth factor receptor beta (PDGFRb) is a key regulator of fibroblasts, pericytes and smooth muscle cells. A high expression of PDGFRb in tumor-associated stroma is associated with worse recurrence free and breast cancer specific survival as well as reduced tamoxifen sensitivity in invasive breast cancer. Although studies indicate that stroma cells can modulate the radiosensitivity of tumor cells, non-leukocytic stroma cells have not been explored as potential predictive markers for radiotherapy (RT) through systematic analyses of clinical samples. In some embodiments, it is the level of stromal PDGFRb that is analyzed in the local cancer tissue sample. In some embodiments, the level of epithelial (e.g., tumor core tissue) PDGFRb is analyzed in the local cancer tissue sample. Without being bound by theory, in some cases, stromal cell infiltrates may contribute to epithelial PDGFRb levels. In some embodiments, reference to “stromal” herein with respect to a tissue sample analyzed for PDGFRb level can include epithelial (e.g., tumor core tissue) and/or stromal.

In some embodiments, PDGFRb is phosphorylated PDGFRb. In some embodiments, PDGFRb is human PDGFRb. In some embodiments, human PDGFRb is a polypeptide having an amino acid sequence of any one of SEQ ID NOs: 1-3, or a mature form thereof. In some embodiments, human PDGFRb is an RNA having a nucleotide sequence that corresponds to any one of SEQ ID NOs: 4-6.

Some embodiments described herein relate to a method for treating a subject for local and/or regional recurrence of invasive breast cancer. A method can comprise one or more of the following steps: a) providing a local cancer tissue sample from a subject who has invasive breast cancer; b) analyzing the local cancer tissue sample for a level of PDGFRb; c) treating the subject with radiotherapy if the local cancer tissue sample has a low level of PDGFRb; and d) treating the subject with an alternative to standard radiotherapy if the local cancer tissue sample has a high level of PDGFRb.

Some embodiments described herein relate to a method of treating a subject. The method can comprise: a) identifying a subject with invasive breast cancer that has a high level of PDGFRb; and b) administering an aggressive breast cancer therapy to the subject locally (e.g., to where there is the high level of PDGFRb). The aggressive breast cancer therapy is at least more than treatment plans that include standard radiation. In some embodiments, this is more than the standard of care defined by the NCCN guidelines, as of October, 2020. In some embodiments, this is more than the standard of care defined by the NCCN guidelines, as of September, 2021.

Some embodiments relate to a method of identifying a subject who will not be adequately responsive to radiation therapy. The method can comprise: a) identifying a subject with invasive breast cancer and b) determining if a local cancer tissue sample from the subject has a high level of PDGFRb. If the local cancer tissue sample from the subject has a high level of PDGFRb, administering an aggressive therapy to the subject, where the aggressive therapy is not standard radiation therapy. In some embodiments, the aggressive therapy is at least: a) standard radiation with the addition of mastectomy or chemotherapy, or b) mastectomy or c) mastectomy and chemotherapy. In some embodiments, this is more than the standard of care defined by the NCCN guidelines, as of October, 2020. In some embodiments, this is more than the standard of care defined by the NCCN guidelines, as of September, 2021.

Some embodiments relate to a method for recommending a treatment to a subject. The method can comprise: a) analyzing a local cancer tissue sample for a level of PDGFRb from a subject; b) recommending that one treats the subject with standard radiotherapy if the local cancer tissue sample has a low level of PDGFRb; and c) recommending that one treats the subject with an alternative to standard radiotherapy if the local cancer tissue sample has a high level of PDGFRb. In some embodiments, the alternative is more than the standard of care defined by the NCCN guidelines, as of October, 2020. In some embodiments, the alternative is more than the standard of care defined by the NCCN guidelines, as of September 2021.

Some embodiments relate to a method for preventing an invasive breast cancer recurrence in a subject. The method can comprise: a) providing a cancer tissue sample from a subject who has invasive breast cancer; b) analyzing the cancer tissue sample for a level of PDGFRb; c) administering standard radiotherapy if the local cancer tissue sample has a low level of PDGFRb; and d) administering an alternative to standard radiotherapy if the local cancer tissue sample has a high level of PDGFRb. In some embodiments, the alternative is more than the standard of care defined by the NCCN guidelines, as of October, 2020. In some embodiments, the standard radiotherapy is that provided in the NCCN guidelines. In some embodiments, the alternative is more than the standard of care defined by the NCCN guidelines, as of September 2021.

Some embodiments relate to a method for preventing a local and/or regional recurrence of an invasive breast cancer in a subject. The method can comprise: a) receiving standard radiotherapy if a local cancer has a low level of PDGFRb; or b) receiving an alternative to standard radiotherapy if the invasive breast cancer has a high level of PDGFRb. In some embodiments, the alternative is more than the standard of care defined by the NCCN guidelines, as of October 2020. In some embodiments, the standard radiotherapy is that provided in the NCCN guidelines. In some embodiments, the alternative is more than the standard of care defined by the NCCN guidelines, as of September 2021.

In some embodiments, the alternative to standard radiotherapy includes, but not limited to, a) standard radiation with the addition of mastectomy or chemotherapy, or b) mastectomy or c) mastectomy and chemotherapy.

Some embodiments relate to a method of modifying a treatment for a subject. The method can comprise: a) identifying a subject with invasive breast cancer that has a high level of PDGFRb; and b) administering a breast cancer therapy to the subject, wherein the breast cancer therapy is more aggressive than a traditional breast cancer therapy, wherein the traditional breast cancer therapy is one recommended for the subject, based on the subject's risk factors excluding PDGFRb levels. In some embodiments, the more aggressive therapy is more aggressive than the standard of care defined by the NCCN guidelines, as of October, 2020. In some embodiments, the more aggressive therapy is more than the standard of care defined by the NCCN guidelines, as of September 2021.

In some embodiments, the traditional and/or standard breast cancer therapy is defined by the NCCN guidelines as of October 2020. The national comprehensive cancer network (NCCN) guidelines are widely recognized and used as the standard for clinical policy in oncology by clinicians and payors. The guidelines are a comprehensive set of guidelines detailing the sequential management decisions and interventions that currently apply to 97 percent of cancers affecting patients in the United States. The NCCN Guidelines provide recommendations based on the best evidence available at the time they are derived. Because new data are published continuously, it is essential that the NCCN Guidelines also be continuously updated and revised to reflect new data and clinical information that may add to or alter current clinical practice standards. Here, in some embodiments, the traditional breast cancer therapy is defined by the NCCN guidelines as of October 2020. In some embodiments, the traditional breast cancer therapy is defined by the NCCN guidelines as of September 2021.

In some embodiments, any of the methods provided herein can take into account the following as “standard” therapies, where the presence or absence of elevated levels of PDGFRb as provided herein can then appropriately modify the therapy (e.g., to avoiding radiotherapy or providing a more aggressive radio therapy):

### Whole Breast Radiation Standard and Aggressive Dosing

Target definition is the breast tissue in entirety.
Radiotherapy (RT) standard dosing:
   1) The whole breast should receive a dose of 45-50.4 Gy in 25-28 fractions or 40-42.5 Gy in 15-16 fractions (hypofractionation is preferred).
   2) A boost to the tumor bed is recommended in patients at higher risk for recurrence. Typical boost doses of a sequential boost are 10-16 Gy in 5-8 fractions. A standard boost can also be given simultaneously integrated (SIB), which means the radiotherapy is given with heterogenous doses to the tumor bed and the remaining breast. A sequential treatment with breast dose fractionation of 2 Gy to 50 to the breast and a sequential boost of eight 2 Gy fractions to the tumor bed approximately corresponds to a simultaneous treatment with 28 fractions with 2,28 Gy to the tumor bed and 1,84 Gy to the remaining breast.
   3) All dose schedules are given 5 days per week.
      Radiotherapy (RT) intensified dosing
   1) In some embodiments, the intensified protocol is to give higher doses in each fraction, e.g. 15 fractions (or more) of 3.2 Gy each, total dose is then 48 Gy (or more), if it is more than would have otherwise been administered to the subject (for an aggressive therapy).
   2) in another embodiment it is to give a boost if it is more than would have otherwise been administered to the subject (for an aggressive therapy).

### Chest Wall Radiation (Including Breast Reconstruction)

The target includes the ipsilateral chest wall, mastectomy scar, and drain sites when indicated.
RT dosing:
   1) Dose is 45-50.4 Gy in 25-28 fractions to the chest wall±scar boost, at 1.8-2 Gy per fraction, to a total dose of approximately 60 Gy. In some embodiments, a total dose of approximately 48-54 Gy can be applied, if it is more than would have otherwise been administered to the subject (for an aggressive therapy).

### Accelerated Partial Breast Irradiation (APBI)

The NCCN Panel accepts the updated 2016 version of the ASTRO APBI
   1) ≥50 years with invasive ductal carcinoma measuring≤2 cm (Ti disease) with negative margin widths of ≥2 mm, no LVI, ER-positive, and BRCA negative; or
   2) low/intermediate nuclear grade, screening-detected DCIS measuring size≤2.5 cm with negative margin widths of ≥3 mm.
RT DOSING
   1) A course of 34 Gy in 10 fractions delivered twice per day is appropriate.

In some embodiments, the standard therapy (which is to then be modified on the basis of the level of PDGFRb) is defined per the NCCN Guidelines® (NCCN Clinical Practice Guidelines in Oncology) Version 6.2020, Sep. 8, 2020, the entirety of which is hereby incorporated by reference, both generally and in specific regard to providing a current standard of care for treatment of subjects having invasive breast cancer. In some embodiments, treating the subject with standard radiotherapy denotes a therapy in line with the guidelines in the NCCN guidelines. In some embodiments, the NCCN guidelines are as of 2020, e.g., as of October 2020. In some embodiments, the standard radiotherapy is as defined by the ASTRO guidelines. In some embodiments, any of the disclosure herein related to NCCN can be modified to instead be directed to ASTRO guidelines. In some embodiments, the standard therapy (which is to then be modified on the basis of the level of PDGFRb) is defined per the NCCN Guidelines® Version 8.2021, Sep. 13, 2021, the entirety of which is hereby incorporated by reference, both generally and in specific regard to providing a current standard of care for treatment of subjects having invasive breast cancer.

In some embodiments, any of the methods provided herein can include a scenario where a low level of PDGFRb denotes a low level of protein expression.

In some embodiments, a low level of PDGFRb denotes a low level of mRNA present in the sample.

In some embodiments, one can examine the levels in the total lysate. In some embodiments, the sample is restricted to stromal. In some embodiments, the sample is stromal and/or epithelial (e.g., tumor core tissue).

In some embodiments, a level of PDGFRb can be determined by any method in the art, including, but not limited to: IHC/immunofluorescence/western blot/laser capture, microdissection, nanostring, PCR, rtPCR, qPCR, deep sequencing, RNA-seq, mRNA micro-array, etc. In some embodiments, one or more detection reagents are used to determine the level of PDGFRb in a sample. Any suitable detection reagent can be used. In some embodiments, suitable detection reagents include, without limitation, an antibody (e.g., monoclonal antibody, polyclonal antibody, etc.) specific to PDGFRb protein, and an oligonucleotide that specifically binds (e.g., hybridizes) to PDGFRb mRNA. In some embodiments, a detection reagent includes a detectable marker. Any suitable detectable marker can be used. In some embodiments, the detectable marker is, without limitation, a fluorescent molecule, a radioisotope, a detectable enzyme. In some embodiments, any method of the present disclosure includes contacting a sample (e.g., a local cancer tissue sample) with a detection reagent to determine the level of PDGFRb in the sample. In some embodiments, whether the sample has a low or high level of PDGFRb is determined by detecting the level and/or localization of the detection reagent that binds specifically to the sample, e.g., by measuring the level and/or localization of the detectable marker in the sample. In some embodiments, the detected level and/or localization of the detection reagent is compared to a suitable control, as provided herein. In some embodiments, the level of PDGFRb determined in a sample is specific to PDGFRb (protein or mRNA). In some embodiments, the level of PDGFRb in a sample does not include the level of PDGFRa in the sample. In some embodiments, a detection reagent (e.g., an antibody to PDGFRb) used to detect the level and/or localization of PDGFRb (e.g., PDGFRb protein) in a sample binds specifically to PDGFRb (e.g., PDGFRb protein) in the sample, and does not bind (e.g., does not bind above background level) to PDGFRa (e.g., PDGFRa protein) in the sample.

In some embodiments, the detection reagent includes an antibody to a PDGFRb protein. Any suitable antibody to PDGFRb protein, e.g., human PDGFRb protein, can be used. In some embodiments, the antibody to PDGFRb binds (e.g., binds specifically) to a polypeptide having an amino acid sequence of any one of SEQ ID NOs: 1-3, or a mature form thereof. In some embodiments, an antibody for detecting PDGFRb in a sample is selected from rabbit monoclonal anti-PDGFRb antibody, clone 28E1, #3169 Cell Signaling, Danvers MA, US; polyclonal goat anti-human PDGFRb, R&D Systems, #AF385; and mouse monoclonal [42G12] to PDGFR beta, Abcam, ab69506. In some embodiments, the antibody to PDGFRb binds (e.g., binds specifically) to a phosphorylated form of the PDGFRb protein. In some embodiments, the antibody to PDGFRb binds (e.g., binds specifically) to an activated form of the PDGFRb protein. In some embodiments, the detection reagent includes an oligonucleotide (e.g., a primer) that binds (e.g., hybridizes) to PDGFRb mRNA, e.g., hybridizes to PDGFRb mRNA under stringent conditions. Any suitable oligonucleotide (e.g., primer) that binds to PDGFRb mRNA, e.g., human PDGFRb mRNA, can be used. In some embodiments, the oligonucleotide (e.g., primer) that binds (e.g., hybridizes) to PDGFRb mRNA binds (e.g., hybridizes) to an RNA having a nucleotide sequence that corresponds to any one of SEQ ID NOs: 4-6.

In some embodiments, a high level of PDGFRb denotes a level, e.g. high level, of protein expression. In some embodiments, a high level of PDGFRb denotes a high level of mRNA present in the sample.

In some embodiments, a high or low level of PDGFRb is determined as a value relative to a control. In some embodiments, the control comprises a positive, a negative, or a positive and a negative control. In some embodiments, the control is an internal control or an external control or both. In some embodiments, normal breast tissue sections can serve as an external control. For example, the periductal stroma of normal breast tissue of women<50 years should display a staining intensity of 3 (0-3). Normal areas within the cancer tissue as well as perivascular cells usually stain strong for PDGFRb and can provide as an internal control. In some embodiments, different cell lines can be used as a positive control for establishing different degrees for scoring (for example 0-3). In some embodiments, different cell lines can be used as a negative control for establishing different degrees for scoring (for example 0-3).

In some embodiments, one can avoid RT (radiotherapy) in selected women. For example, the baseline risk in elderly women with small, node-negative, hormone receptor-positive tumors may be low enough that some may reasonably opt to avoid RT and its associated risks and toxicities in this population. NCCN guidelines indicate that it may be reasonable to avoid RT in selected older women with estrogen receptor (ER)-positive, HER2-negative breast cancer. Specifically, this includes women aged 65 years or older with clinically node-negative, small (T size<3 cm) breast cancer who are willing to initiate adjuvant endocrine therapy. It is recognized, however, that chronologic age may differ from “biological age,” and that some women with small, node-negative tumors, who are older than 65 with good baseline health may have a higher likelihood of benefit from RT than a younger patient with significant comorbidities. Moreover, other factors to consider, in addition to patient age and size of tumor, include high tumor grade, lymphovascular invasion, or low intensity of ER expression, all of which increase the rates of local failure and may make RT more desirable. In some situations, the decision to omit RT can take into account potential comorbidities and tumor features that could affect long-term survival. Patients may understand that without RT, the rate of in-breast recurrence may be higher over time and the rate of requiring subsequent mastectomy may be higher. Moreover, compliance with endocrine therapy is an important aspect of treatment, particularly for those in whom RT was omitted. Given the above, in some embodiments, one will want to use the present PDGFRb analysis in excluding radiation therapy for older women with estrogen receptor (ER)-positive, HER2-negative breast cancer, when the expected benefit of RT is low such that RT may be omitted without meaningfully increasing the risk of recurrence. In some embodiments, one will want to use the present PDGFRb analysis in excluding radiation therapy when the expected benefit of RT is low such that RT may be omitted without meaningfully increasing the risk of recurrence, and endocrine therapy or systemic chemotherapy in combination or individually without radiation therapy. In some embodiments, one will want to use the present PDGFRb analysis in excluding radiation therapy when the expected benefit of RT is low such that RT may be omitted without meaningfully increasing the risk of recurrence, using mastectomy without radiation therapy instead of mastectomy with radiation therapy or breast conserving surgery with radiation therapy. In some embodiments, one will want to use the present PDGFRb analysis in excluding radiation therapy when the expected benefit of RT is low such that RT may be omitted without meaningfully increasing the risk of recurrence, using breast conserving surgery without radiation therapy instead of breast conserving surgery with radiation therapy.

In some embodiments, the control includes a level defined to one or more reference genes, so called housekeeping genes. In some embodiments, the housekeeping genes that may be used are selected from: CCSER2, SYMPK, ANKRD17 and PUM1, PUM1 and RPL13A, DDX5, LAPTM4A, P4HB, and RHOA. In some embodiments, the housekeeping genes that may be used are selected from: ACTB, ALAS1, B2M, CDKN1A, G6PD, GAPDH, GUSB, HBB, HMBS, HPRT1, HSP90AB1, IPO8, LDHA, NONO, PGK1, POP4, PPIA, PPIH, PSMC4, PUM1, RPL13A, RPL30, RPLPO, RPS17, RPS18, SDHA, TBP, TFRC, UBC, YWHAZ, TUBB, RPN1. In some embodiments, the housekeeping genes that may be used are selected from: PUMLIP08, UBC, ACTB, and RPN1. In some embodiments, the housekeeping genes that may be used are selected from: MBTPS1, HNRNPAO, SF3A1, SF3B2, GGNBP2, HNRNPUL2, SFRS3, RTF1, CIAO1, TM9SF3 and SFRS4. In some embodiments, the reference genes may be selected by analyzing TCGA transcriptome sequencing data in order to evaluate key characteristics needed for reference genes, including the expression stability and absence of correlation between their mRNA. Both widely used reference genes familiar to one skilled in the art and other reference gene candidates may be evaluated from TCGA using tumor and matched normal tissues. Reference genes may be selected from analysis of TCGA sets containing matched Tumor and normal tissue including: BRCA, LUAD, LUSC, KIRP, PRAD, COAD, HNSC LIHC, STAD, THCA, and BLCA. Krasnov G S, Kudryavtseva A V, Snezhkina A V, et al. Pan-Cancer Analysis of TCGA Data Revealed Promising Reference Genes for qPCR Normalization. Front Genet. 2019; 10:97. Published 2019 Mar. 1. doi:10.3389/fgene.2019.00097.

In some embodiments, the high or low levels of PDGFRb are defined by a comparison of PDGFRb levels from local tissue sample to a control sample in a healthy subject.

In some embodiments, the high or low levels of PDGFRb are defined by a comparison of PDGFRb levels from the local tissue sample to a control sample from a tissue in the subject that does not include invasive cancer.

In some embodiments, the high or low level of PDGFRb are defined by a comparison to a standardized level set by a level of expression of a house keeping gene.

In some embodiments, treating the subject with an alternative to standard radiotherapy denotes either a) administering a more intense level of therapy than that outlined in the NCCN guidelines, or b) the same NCCN guideline modalities given concurrently, e.g. RT+chemotherapy, RT+targeted therapies. In some embodiments, this can include systemic therapies. In some embodiments, this can include anti-HER2 therapy, immunotherapy, PARP inhibitor therapy, and/or endocrine therapy for example. In some embodiments, a radiation boost with higher dose levels or with broader indications than in current NCCN guidelines, mastectomy, concurrent radiochemotherapy can be employed.

Examples of protocols for intensified radiotherapy treatment (aggressive therapy) include (but are not limited to) an initial treatment step comprising one of the following: 1) 25 (or more) fractions of 2 Gy each (total 50 Gy or more); 2) 15 (or more) fractions of 2.67 Gy (total 40 Gy or more); 3) 16 (or more) fractions of 2.66 Gy (total 42,5 Gy or more); 4) 5 (or more) fractions of 5.2 Gy (total 26 Gy or more).

In some embodiments, the initial treatment step is followed by an addition of 10-16 Gy (or more) administered in 2 Gy fractions or brachytherapy of 10-15 Gy (or more).

In some embodiments, the intensified protocol (aggressive therapy) is to give a concomitant boost with higher doses in each fraction, e.g. 15 fractions (or more) of 3.2 Gy each, total dose is then 48 Gy (or more), to a volume corresponding to the area of the site of the removed tumor, while the dose to the remaining part of the breast is 40 Gy in 15 fractions of 2.67 Gy each.

In some embodiments, the intensified protocol (aggressive therapy) is to give a concomitant boost with higher doses in each fraction, e.g. 28 fractions of 2.25 Gy each, total dose is then 63 Gy, to a volume corresponding to the area of the site of the removed tumor, while the dosse to the remaining part of the breast is 28 fractions of 1.84 Gy each to a total dose of 51.52 Gy.

In some embodiments, the NCC (or NCCN) guidelines indicate that a RT boost to the tumor bed is intended to decrease locoregional recurrence rates. While RT to the tumor bed following breast-conserving surgery and WBRT is recommended in younger women, its routine use in older women is less clear. In some embodiments, when a boost is recommended to an older woman with stage 1 luminal phenotypes, for whom it is optional, but in the case where PDGFRb is high, they need not administer standard radiotherapy.

In some embodiments, patient receive an RT boost after WBRT, except for selected women aged 60 and older with stage 0 to I luminal phenotypes resected with negative margins, for whom it is optional. The degree of benefit and the associated potential skin toxicities following a boost in patients who had received hypofractionated RT can be unclear. The decision to give a boost in these patients should be made after a discussion between the patient and the treating radiation oncologist. Thus, the present use of PDGFRb can be used in assisting subjects for whom treatment is optional, into a stronger position for either taking it (if it may have a greater chance of working) or not taking it (if there is a lower chance of it working for them). In some embodiments, if an RT boost is administered, 10 to 14 Gy in either 2 Gy or 2.5 Gy fractions, it is usually administered, with a boost dose, in part, dependent upon the dose and fractionation delivered to the whole breast. In some embodiments, a more aggressive therapy will deliver more than the standard boost as identified in NCCN or ASTRO guidelines. For example a boost total of more than 10 Gy in 5 fractions, more than 14 Gy in boost with at least a standard whole breast dose. In some embodiments, a simultaneous integrated boost can be employed.

In some embodiments, IORT at 20 Gy can be administered to the cavity and 45-50 Gy can be administered to the whole breast (e.g., for an aggressive therapy). In some embodiments, the amount can be 10, 15, 20, 25, 30 Gy or more for IORT and then an amount administered to the whole breast as well sufficient to make it an aggressive therapy (e.g., at least 55, 50, 45, 40, 35 Gy). In some embodiments, more than 60 Gy can be administered in total to the target. In some embodiments, 64 Gy administered in total to the target. In some embodiments, more than 61, 62, 63, 64, 65, 66, 67, 68, 69, 70, 71, 72, 73, 74, 75, 76, 77, 78, 79, 80, or more Gy can be administered to the subject as an aggressive therapy (e.g., when PDGFRb levels are high).

In some embodiments, treating the subject with an alternative to standard radiotherapy denotes applying radiotherapy at an intensity of more than that noted above. In some embodiments, the radiation therapy applied is at least 25 fractions of 2 Gy each (total 50 Gy), 15 fractions of 2.67 Gy each (total 40 Gy), 16 fractions of 2.66 Gy each (total 42.5 Gy) or 5 fractions of 5.2 Gy each (total 26 Gy), for example. In some embodiments, an example of a protocol for intensified radiotherapy treatment include at least an initial treatment step comprising one of the following: 1) 25 fractions of 2 Gy each (total 50 Gy); 2) 15 fractions of 2.67 Gy (total 40 Gy); 3) 16 fractions of 2.66 Gy (total 42,5 Gy); or 4) 5 fractions of 5.2 Gy (total 26 Gy). The initial treatment (e.g. one of points 1-4 above) step is followed by an addition of 10-16 Gy administered in 2-2.5 Gy fractions or brachytherapy of 10-15 Gy. In some embodiments, an aggressive therapy is to give a concomitant boost with higher doses in each fraction, e.g. 15 fractions of 3.2 Gy each, total dose is then 48 Gy, to a volume corresponding to the area of the site of the removed tumor, while the dose to the remaining part of the breast is 40 Gy in 15 fractions of 2.67 Gy each. In some embodiments, an aggressive therapy is to give a concomitant boost with higher doses in each fraction, e.g. 28 fractions of 2.25 Gy each, total dose is then 63 Gy, to a volume corresponding to the area of the site of the removed tumor, while the dose to the remaining part of the breast is 28 fractions of 1.84 Gy each to a total dose of 51.52 Gy.

In some embodiments, treating the subject with an alternative to standard radiotherapy denotes applying radiotherapy in combination with at least one of the following: chemotherapy, anti-HER2 therapy, endocrine therapy, immunotherapy, PARP inhibitor therapy, or other targeted therapies such as tyrosine kinase inhibitors or monoclonal antibodies against PDGFRb.

In some embodiments, high PDGFRb denotes the subject who has PDGFRb levels at the highest quartile of a population of PDGFRb levels of a population of people from the SweBCG91RT clinical trial of 1991-97. In some embodiments, the SweBCG91RT can be tested/grouped into tertiles or have a median cut off for PDGFRb score. The patient group in the highest tertile as well as those patient with PDGFRb score>median will not show benefit from radiotherapy. In some embodiments, the population of people from the SweBCG91RT trial has clinicopathological characteristics as provided in Table 3 of FIG. 7. The population of people from the SweBCG91RT trial has been characterized in Malmstrom et al. (1990) Eur J Cancer Oxf Engl 39:1690-1697; and Sjostrom et al. (2017) J Clin Oncol 35:3222-3229, each of which is incorporated by reference in its entirety.

In some embodiments, PDGFRb levels are obtained by multiplication of an intensity score and positive fraction score. The score is divided into three groups, each group as a tertile. Patients of the highest tertile (scores 6-12) are assigned “PDGFRb high” status. The combination of the lowest and middle tertiles is designated “PDGFRb low” status. In some embodiments, a high or low PDGFRb level is determined by a combination of a) intensity of staining and b) percent of positive fraction of the tumor stained, wherein greater amounts in either a) or b) result in an increased PDGFRb level. In some embodiments, a) is defined into four equal parts (0-3), and b) is defined into five parts (0-4). In some embodiments, the score is determined by multiplying a) and b) together. In some embodiments, the highest ⅓ of the population has a “high” level of PDGFRb, and/or the score is 6-12.

In some embodiments, the cutoff can instead be limiting to the top: 10, 15, 25, 30% of the population.

In some embodiments, the score for high risk can be: anything above 6, 6 or higher, 8 or higher, 9 or higher, 10 or higher, 11 or higher. In some embodiments, fractional scores are also permitted for one or both of a) and b), and thus, can be 0.1, 0.2, 0.3, 0.4, 0.5, 0.7. 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 (as appropriate ranges 0-3 for a) and 0-4 for b).

Thus, in some embodiments, subjects having PDGFRb staining that is at least average or higher intensity (2 or 3), and at least having average or higher percentage of staining (2-4), can result in a high level of PDGFRb (acknowledging that there is a small group that could be 2 on a) and 2 on b) and thus result in a score of 4, which would not be high risk).

In some embodiments, any of the methods provided herein can be performed via computer assisted scoring and in some embodiments provide further refinement of the scoring resolution. In some embodiments, the scoring can take into account a pathology review as well. In some embodiments, PDGFRb intensity and positive stroma fraction can be determined in a defined number of high power/vision fields per patient case and then the average intensity and positive fraction for the given patient will be calculated. In some embodiments, one can employ a pathology-based definition for the area to be scored for PDGFRb, which can be defined broadly as “tumor associated stroma”. In some embodiments, this can be specified e.g. tumor area+two high power fields outside of tumor (cell)border. In some embodiments, one can analyze tissue microarray cores. In some embodiments, one can analyze full sections.

In some embodiments, one can define the scoring to be based on dividing the percentage positivity into 0-10, e.g. 10% increments of 100% possible. In some embodiments, one can define the scoring to be based on dividing the percentage positivity into 0-20, e.g. 5% increments of 100% possible. In some embodiments, one can define the scoring to be based on dividing the percentage positivity into 0-100, e.g. 1% increments of 100% possible. In some embodiments, these alternative embodiments can be scaled to be equivalent to the scoring using the 0-12 point scale.

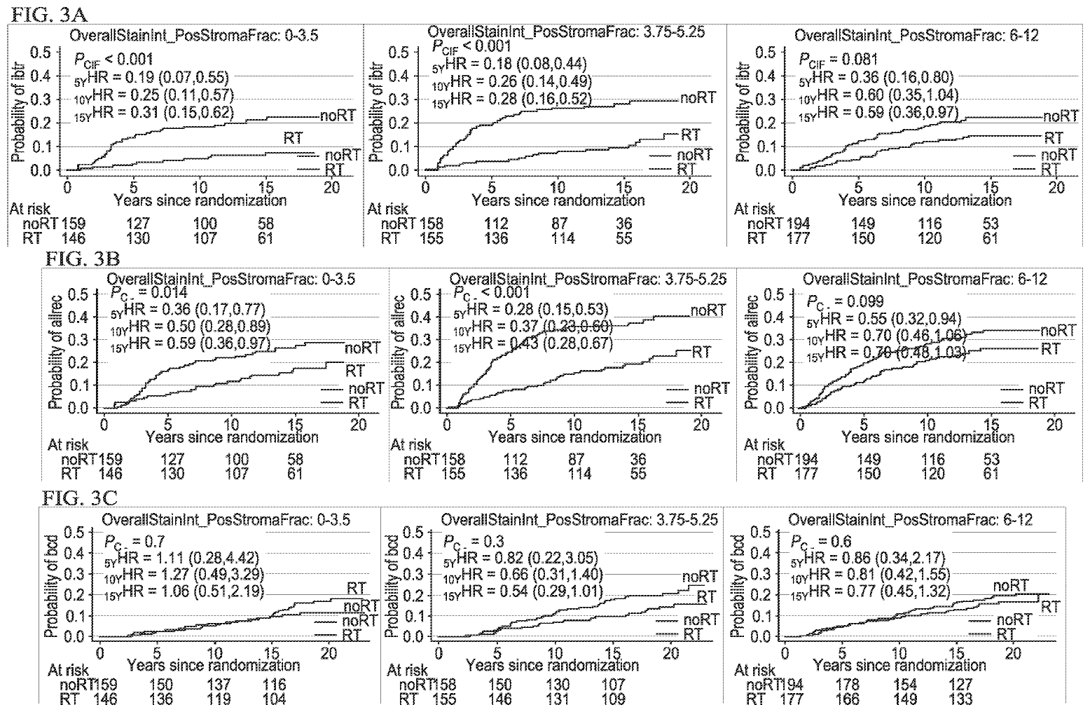

In some embodiments, the cutoff of high vs. low is defined as follows: the score is obtained by multiplication of the intensity score and positive fraction score, and the obtained scores were split in tertiles; patients of the highest tertile (scores 6-12) are assigned “PDGFRb high status”; as noted in FIGS. 3A-3C in the Example 8 below. Exemplary cut-off points for the tertiles are stated therein. In some embodiments, the lowest and middle tertile can be combined to the final designated “PDGFRb low” group.

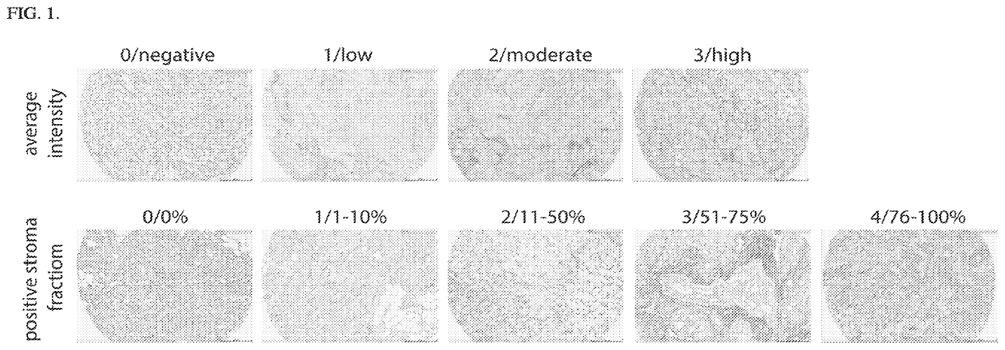

In some embodiments, the scoring can be in line with that shown in Example 8. In some embodiments, the scoring can employ the images in FIG. 1 to define the scoring categories. In some embodiments, the scoring can be as follows: a) stromal PDGFRb staining an average intensity following a four-grade scale (0/negative; 1/low; 2/moderate; 3/high) and b) positive stroma fraction as well as overall stroma abundance following a five-grade scale (0/0%; 1/1-10%; 2/11-50; 3/51-75%; 4/76-100%) (See FIG. 1 for example). In some embodiments, the overall stroma fraction (b)) can be rated on a five-grade scale (0/0%; 1/1-10%; 2/11-50; 3/51-75%; 4/76-100%). In some embodiments, these categories and accompanying examples and figures can be normalized to other controls, or used as controls, for any of the methods provided herein. In some embodiments, the staining and scoring in the categories provided in FIG. 1, can be used for the creation of positive or negative controls for comparison for any subject to be scored.

In some embodiments, the level of PDGFRb is analyzed using “hot spot” scoring. In some embodiments, hot spot scoring includes measuring the stromal PDGFRb staining intensity in the area of the tumor-associated stroma displaying highest PDGFRb expression. In some embodiments, hot spot scoring includes measuring the epithelial PDGFRb staining intensity in the area of the tumor-associated epithelia displaying highest PDGFRb expression.

In some embodiments, the scoring system is such that “high” PDGFRb denotes the subject who has PDGFRb levels at the highest 10% of a population of PDGFRb levels of a population of people. In some embodiments, “high” can encompass the top 33, 30, 25, 20, 15, 10, 5, or 1% of the population for staining of invasive breast cancer samples.

In some embodiments, the scoring system is such that “high” PDGFRb denotes the subject who has PDGFRb levels at the highest ⅓rd of a population of PDGFRb levels of a population of people. Similarly, a low PDGFRb amount is one that is at the lower ⅔rds of the population. In some embodiments, this is determined by analyzing protein levels and/or mRNA levels.

In some embodiments, the scoring system is such that “high” PDGFRb denotes the subject who has PDGFRb levels at the highest ¼th of a population of PDGFRb levels of a population of people. Similarly, a low PDGFRb amount is one that is at the lower ¾th of the population. In some embodiments, this is determined by analyzing protein levels and/or mRNA levels.

In some embodiments, the scoring system is such that “high” PDGFRb denotes the subject who has PDGFRb levels at the highest ⅕ th of a population of PDGFRb levels of a population of people. Similarly, a low PDGFRb amount is one that is at the lower ⅘ th of the population. In some embodiments, this is determined by analyzing protein levels and/or mRNA levels.

In some embodiments, the scoring system is such that “high” PDGFRb denotes the subject who has PDGFRb levels at the highest 1/10 th of a population of PDGFRb levels of a population of people. Similarly, a low PDGFRb amount is one that is at the lower 9/10th of the population. In some embodiments, this is determined by analyzing protein levels and/or mRNA levels.

In some embodiments, the scoring system is such that “high” PDGFRb denotes the subject who has PDGFRb levels at the highest ¼ th of a population of PDGFRb levels of a population of people. Similarly, a low PDGFRb amount is one that is at the lower ¼th of the population. In some embodiments, this is determined by analyzing protein levels and/or mRNA levels.

In some embodiments, any of the scoring system provided herein can be based on the distribution of individuals in the population of people from the SweBCG91RT trial. In some embodiments, any fraction or percentage used to classify expression of PDGFRb as high or low can be derived by using the population of people from the SweBCG91RT trial as a guide or reference. In some embodiments, the population of people used in any of the scoring systems provided herein is the population of people from the SweBCG91RT trial.

In some embodiments, the high or low PDGFRb is determined as a function of staining of the fraction of whole stroma, epithelia (e.g., tumor core) or both, that is positive and a level of expression. In some embodiments, the fraction of staining is multiplied by the level of expression.

In some embodiments, a level of PDGFRb is analyzed as a continuous metric so that a continuous risk assessment is further provided to the subject.

In some embodiments, a level of PDGFRb is analyzed as a continuous metric so that a continuous risk assessment is expressed as risk of not-responding to RT provided to the subject.

In some embodiments, a level of PDGFRb is analyzed as a continuous metric so that a continuous risk assessment is expressed as RT benefit provided to the subject.

In some embodiments, a method of treating a subject is provided. The method can comprise: a) identifying an incremental risk to a subject of a local recurrence of an invasive breast cancer based on a level of PDGFRb in a sample of an invasive breast cancer in the subject; and b) administering an aggressive breast cancer therapy to the subject based upon the incremental risk, wherein a higher incremental risk will increase: i) a likelihood of an aggressive breast cancer therapy that is at least more than what would be recommended by the NCCN; ii) the aggressiveness of the aggressive breast cancer; or iii) both i) and ii).

In some embodiments, a method of treating a subject is provided. The method can comprise: a) identifying an incremental benefit from RT to a subject of a local recurrence of an invasive breast cancer based on a level of PDGFRb in a sample of an invasive breast cancer in the subject; and b) administering an aggressive breast cancer therapy to the subject based upon the incremental RT benefit, wherein a lower incremental RT benefit will increase: i) a likelihood of an aggressive breast cancer therapy that is at least more than what would be recommended by the NCCN; ii) the aggressiveness of the aggressive breast cancer; or iii) both i) and ii).

In some embodiments, a method of treating a subject is provided. The method can comprise: a) identifying an incremental benefit from RT to a subject of a local recurrence of an invasive breast cancer based on a level of PDGFRb in a sample of an invasive breast cancer in the subject; and b) administering an aggressive breast cancer therapy to the subject based upon the incremental RT benefit, wherein a lower incremental RT benefit will increase: i) the likelihood of an insufficient increase in locoregional risk reduction to merit standard radiation therapy recommended by the NCCN, and recommending to omit radiation therapy.

In some embodiments, any of the methods provided herein can be applied to a newly diagnosed tumor in the breast, and thus can be a method for treating a subject for a breast tumor, said method comprising: providing a tissue sample from a subject who has breast cancer. One can then analyze the tissue sample for a level of stromal PDGFRb. In some embodiments, one can analyze the tissue sample for a level of epithelial PDGFRb. One can then treat the subject with radiotherapy if the sample has a low level of PDGFRb. One can then treat the subject with an alternative to standard radiotherapy if the tissue sample has a high level of PDGFRb.

### EXAMPLES

### Example 1

This non-limiting example describes a method for treating a subject for local and/or regional recurrence of invasive breast cancer.

A local cancer tissue sample is taken from a subject who has invasive breast cancer. RNA sample are extracted from the tissue sample. PDGFRb mRNA levels are measured and scored. The subject's PDGFRb level is assigned a “PDGFRb high” status after comparing with a control sample and receiving a score of 6 or higher. One then treats the subject with an alternative to standard radiotherapy, such as a more intense form of radiotherapy or additional therapies in combination to radiotherapy, etc.

### Example 2

This non-limiting example describes a method of treating a subject.

A local cancer tissue sample is taken from a subject who has invasive breast cancer. RNA samples are extracted from the tissue sample. PDGFRb mRNA levels are measured and scored. The subject's PDGFRb level is assigned a “PDGFRb high” or “PDGFRb low” status after comparing with control sample. One thereby identifies a subject with invasive breast cancer that has a high level of PDGFRb. One can then administer an aggressive breast cancer therapy (beyond the recommendation provided in the NCCN guidelines as of September, 2020) to the subject.

### Example 3

This non-limiting example describes a method of identifying a subject who will not be adequately responsive to radiation therapy.

Firstly, a subject with invasive breast cancer is identified. A local cancer tissue sample is taken from the subject who has invasive breast cancer. Protein and/or RNA sample are extracted from the tissue sample. PDGFRb protein or mRNA levels are measured and scored. The subject's PDGFRb level is assigned a “PDGFRb high” or “PDGFRb low” status (scoring 0-12, with 6-12 being high) and can be normalized by a comparison with a control sample. If the local cancer tissue sample from the subject has a high level of PDGFRb, one administers an aggressive therapy to the subject, where the aggressive therapy is beyond the standard radiation therapy, and the aggressive therapy is at least: a) standard radiation with the addition of mastectomy or chemotherapy, or b) mastectomy or c) mastectomy and chemotherapy.

### Example 4

This non-limiting example describes a method for recommending a treatment to a subject.

A local cancer tissue sample is taken from a subject who has cancer. Protein and/or RNA sample are extracted from the tissue sample. PDGFRb protein or mRNA levels are measured and scored. The subject's PDGFRb level is assigned a “PDGFRb high” or “PDGFRb low” status and can be compared with control sample. A high score is 6-12, and a low score is lower than 6. Recommending that one treats the subject with standard radiotherapy if the local cancer tissue sample has a low level of PDGFRb; and that one treats the subject with an alternative to standard radiotherapy if the local cancer tissue sample has a high level of PDGFRb. If the subject has a high level of PDGFRb, then one treats with an alternative to radiotherapy. If the subject has a low level of PDGFRb, then one treats with standard radiotherapy.

### Example 5

This non-limiting example describes a method for preventing an invasive breast cancer recurrence in a subject.

A local cancer tissue sample is taken from a subject who has invasive breast cancer. Protein and/or RNA sample are extracted from the tissue sample. PDGFRb protein or mRNA levels are measured and scored. The subject's PDGFRb level is assigned a “PDGFRb high” or “PDGFRb low” status (a score of up to 12) after being compared with control sample. Administering standard radiotherapy if the local cancer tissue sample has a low level of PDGFRb; and administering an alternative to standard radiotherapy if the local cancer tissue sample has a high level of PDGFRb.

### Example 6

This non-limiting example describes a method for preventing a local and/or regional recurrence of an invasive breast cancer in a subject.

A local cancer tissue sample is taken from a subject who has invasive breast cancer. Protein and/or RNA sample are extracted from the tissue sample. PDGFRb protein or mRNA levels are measured and scored. The subject's PDGFRb level is assigned a “PDGFRb high” or “PDGFRb low” status after compared with control sample (a score of up to 12). Receiving standard radiotherapy if a local cancer has a low level of PDGFRb (less than 6); or receiving an alternative to standard radiotherapy if the invasive breast cancer has a high level of PDGFRb. The alternative to standard radiotherapy includes, but not limited to, a) standard radiation with the addition of mastectomy or chemotherapy, or b) mastectomy or c) mastectomy and chemotherapy. The standard radiotherapy is that provided in the 6.2020 NCCN Guidelines®, as of September 2020.

### Example 7

This non-limiting example describes a method of modifying a treatment for a subject.

A local cancer tissue sample is taken from a subject who has invasive breast cancer. Protein and/or RNA sample are extracted from the tissue sample. PDGFRb protein or mRNA levels are measured and scored. The subject's PDGFRb level is assigned a “PDGFRb high” or “PDGFRb low” status (a score of up to 12). A subject with invasive breast cancer that has a high level of PDGFRb is identified. Administering a breast cancer therapy to the subject, wherein the breast cancer therapy is more aggressive than a traditional breast cancer therapy (as defined by the NCCN September 2020 guidelines), wherein the traditional breast cancer therapy is one recommended for the subject, based on the subject's risk factors excluding PDGFRb levels.

### Example 8

The present non-limiting Example details a scoring analysis of various embodiments provided herein.

Radiotherapy (RT) in combination with breast conserving surgery (BCS) is currently the preferred treatment over mastectomy for patients with early stage breast cancer. Nevertheless, a minority of these patients will suffer from local recurrences during the first decade after surgery. Classic histopathological variables are unable to identify patients with different proportional benefits from adjuvant RT. An increasing focus is being put on the microenvironment as a modulator of the benefit from adjuvant RT.

The role of stromal PDGFRb expression in progression and treatment response of invasive breast cancer is still not fully understood. A high expression of PDGFRb in the tumor stroma has been associated with unfavorable clinicopathological variables and shorter recurrence free and breast cancer specific survival, univariably, in a population-based cohort although there are also studies which have failed to confirm the prognostic effect.

The current literature is conflicting regarding the function of stromal PDGFRb on prognosis as well as treatment response in invasive breast cancer. The prognostic and predictive impact of stromal PDGFRb on ipsilateral breast tumor recurrence (IBTR), any recurrence and breast cancer specific death (BCSD) was analyzed in a large and clinically well-annotated randomized RT trial of early stage breast cancer patients.

### Marker Staining

The Ventana Benchmark Discovery autostainer system (NexES V10.6) was used for immunohistochemical staining of PDGFRb on 4 um freshly cut sections from formalin-fixed paraffin embedded tissue-microarray (TMA) blocks. The protocol included extended antigen retrieval with pH10 Tris buffer (Sigma-Aldrich and Merck Kgaa, Darmstadt, Germany) and incubation for 1 hour at 37° C. with the primary antibody (rabbit monoclonal anti-PDGFRb antibody, clone 28E1, #3169 Cell Signaling, Danvers MA, US) diluted at 1:100 dilution in Discovery Antibody Diluent (Ventana, Tucson, Arizona, US). Chromogenic detection was performed using the Discovery OmniMap anti-rabbit HRP (RUO) kit (Ventana) with secondary antibody incubation for 32 minutes at room temperature. Hematoxylin II was applied for 10 minutes and subsequent bluing for 6 minutes (Ventana) in order to obtain counterstaining. Staining procedure was established by C. Strell.

Antibody-based cross detection of the structurally related PDGFRa was excluded as described previously [1].

1. Strell C, Paulsson J, Jin S-B, Tobin N P, Mezheyeuski A, Roswall P, et al. Impact of Epithelial-Stromal Interactions on Peritumoral Fibroblasts in Ductal Carcinoma in Situ. J Natl Cancer Inst. 2019; 111:983-95.

### Marker Evaluation

The stained slides were scanned for evaluation (PathXL, Belfast, Northern Ireland). The scoring of stromal PDGFRb staining was performed blinded by two independent raters for average intensity following a four-grade scale (0/negative; 1/low; 2/moderate; 3/high) and positive stroma fraction as well as overall stroma abundance following a five-grade scale (0/0%; 1/1-10%; 2/11-50; 3/51-75%; 4/76-100%) (See FIG. 1). Furthermore, the overall stroma fraction was rated on a five-grade scale (0/0%; 1/1-10%; 2/11-50; 3/51-75%; 4/76-100%). TMAs included two cores of 1.0 mm diameter per patient. The degree of scoring consistency between raters was evaluated using unweighted Cohen's kappa (κ) correlation. Rare cases for which the scores of the raters differed by more than two grades were reevaluated to exclude technical errors. The evaluations of both raters were averaged, and the product between PDGFRb staining intensity and positive stroma fraction was calculated.

### Test Material

The retrospective analysis included patients from the SweBCG91RT trial who have been described elsewhere [2,3] (Table 3 in FIG. 7). In short, 1178 lymph-node negative (N0) patients with stage I or IIA breast cancer were randomly assigned to BCS with or without whole-breast RT between the years 1991 and 1997 and followed for a median time of 15.2 years (FIG. 6). Tumor blocks from initial surgery were retrieved, and tumors were classified according to the St Gallen International Breast Cancer Conference Expert Panel 2013 using immunohistochemical panels. ER and HER2 evaluation has been described previously. In brief, the cutoff used to consider a tumor ER positive was 1%, for PgR the cutoff was ≥20% to distinguish luminal A-like from luminal B-like tumors. Triple negative tumors were defined as negative for ER, PgR and HER2. HER2 was considered positive if 3+ on immunohistochemistry level or amplified on silver in situ hybridization. Patients were well balanced regarding clinicopathological baseline characteristics across the treatment arms.

2. MaInnstronn P, Holmberg L, Anderson H, Mattsson J, Jonsson P E, Tennvall-Nittby L, et al. Breast conservation surgery, with and without radiotherapy, in women with lymph node-negative breast cancer: a randomised clinical trial in a population with access to public mammography screening. Eur J Cancer Oxf Engl 1990. 2003; 39:1690-7.
3. Sjostronn M, Lundstedt D, Hartman L, Holmberg E, Killander F, Kovacs A, et al. Response to Radiotherapy After Breast-Conserving Surgery in Different Breast Cancer Subtypes in the Swedish Breast Cancer Group 91 Radiotherapy Randomized Clinical Trial. J Clin Oncol. American Society of Clinical Oncology; 2017; 35:3222-9.

### Statistics

A statistical analysis was performed. Time to ipsilateral tumor recurrence (IBTR) as first event within 10 years was used as primary endpoint. Secondary endpoints were time to any breast cancer recurrence within 10 years (allrec: IBTR, regional recurrence or distal recurrence) and time to breast cancer specific death (BCSD) within 15 years. Regional recurrence, distant recurrence and death were considered competing risks for IBTR.

Known clinical variables were included in multivariable analysis: age group, histological grade, subtype and radiotherapy (RT) treatment. Subtype was kept in multivariable analysis, despite not being significant in univariable analysis, because of the biologic relevance. Hazard ratios (HRs) were calculated with cause-specific Cox proportional hazards regression to reflect the biologic effect of RT in the presence of competing risks. Correlation analysis between clinicopathologic parameters and stromal PDGFRb status was tested using Spearman's Rank test.

Figures of cumulative incidence were created. p values for the hazard ratio between compared groups were denoted Par in the plots. p values<0.05 were considered significant. For the final analysis PDGFRb scoring data was split in tertiles, as predefined, and referred to as PDGFRb low (n=305), medium (n=313) or high (n=371) score group. STATA 15.1 was used for analysis (StataCorp. 2017. Stata: Release 15. Statistical Software. College Station, TX: StataCorp LLC).

The proportional hazards assumption was checked graphically and tested with Schoenfeldt's test. It was violated for RT, histological grade, subtype and RT: PDGFRb score and these values should thus be interpreted as the mean value over 10 years.

### Results

### Summary of the Results

Trends for a reduced effect of radiotherapy benefit in PDGFRb high group was detected. A higher PDGFRb score conferred an increased risk of any recurrence, which partly can be explained by its association with estrogen receptor negativity and young age.

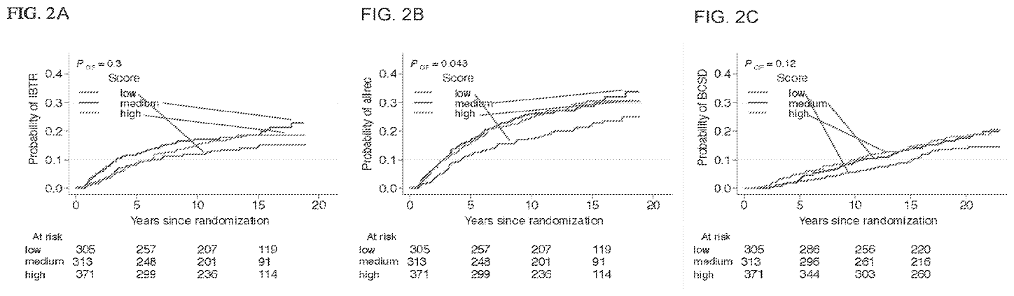

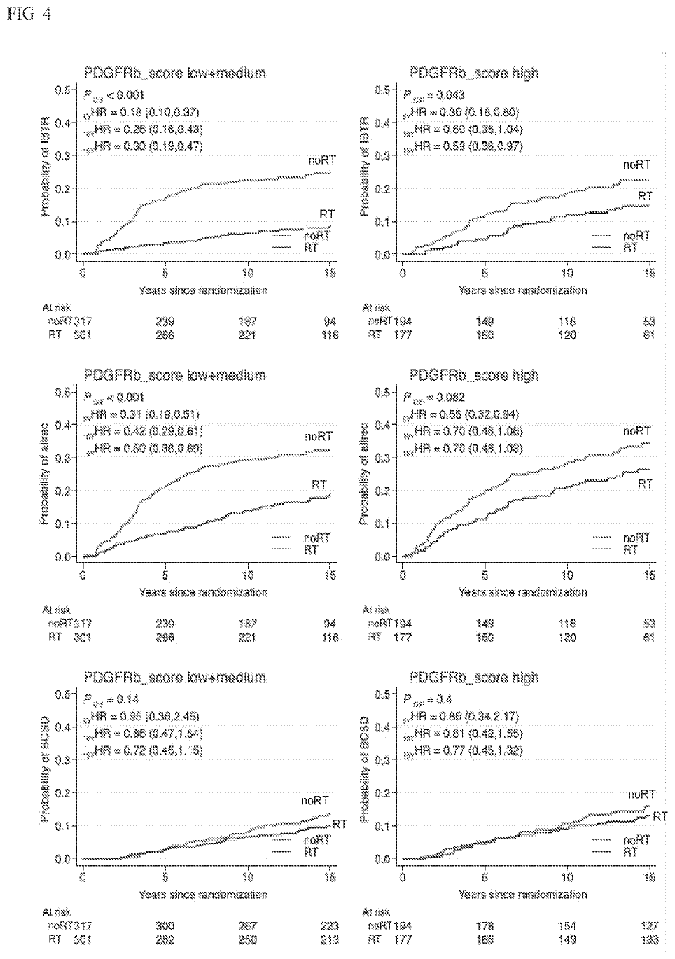

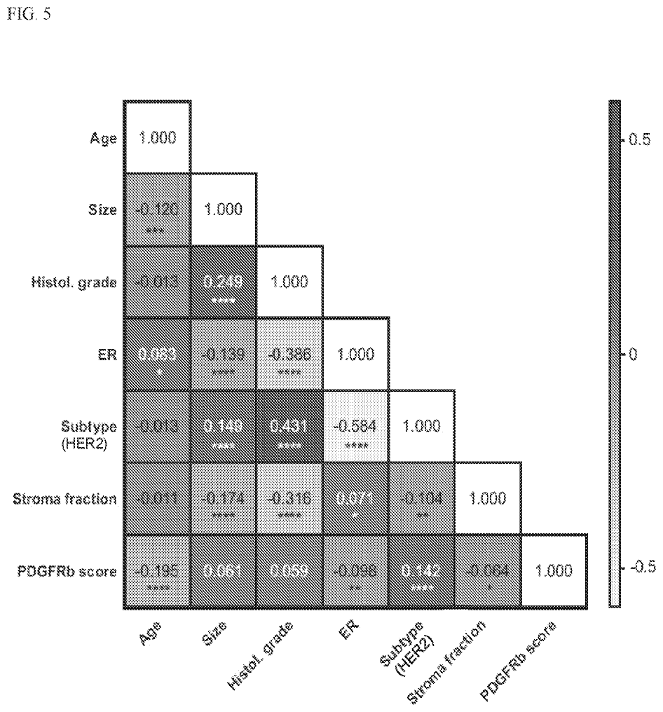

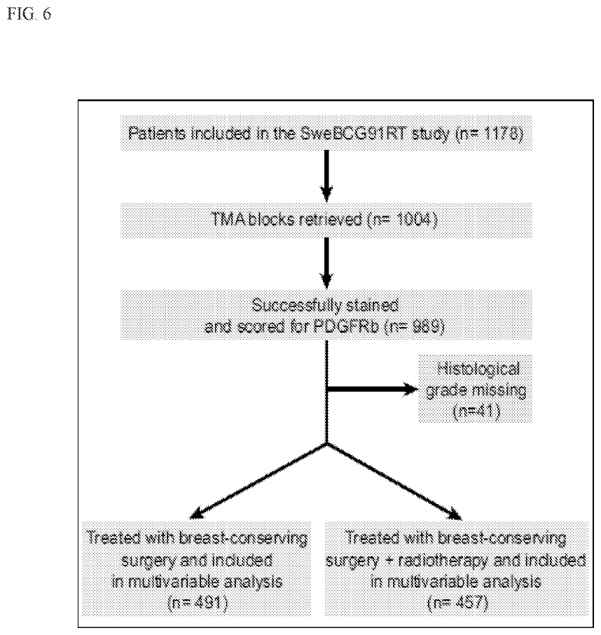

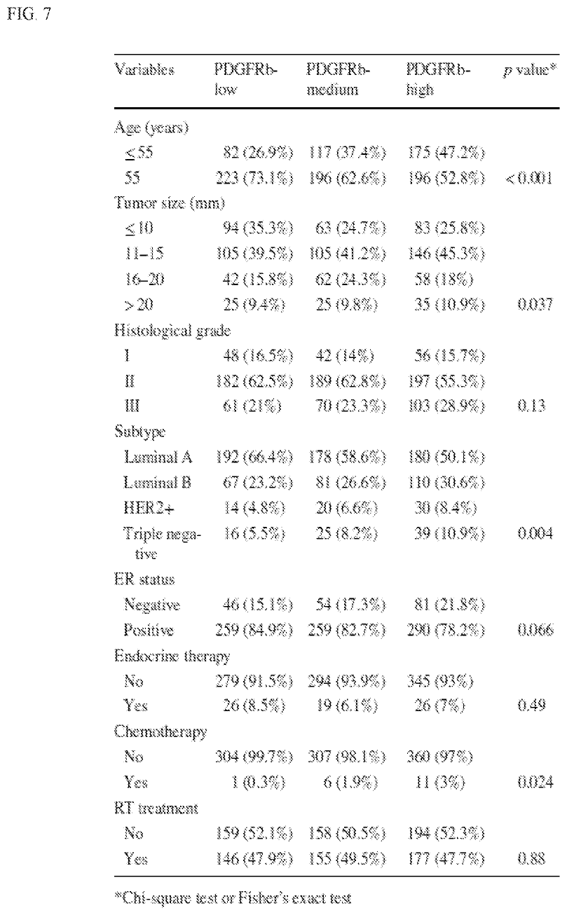

The results are shown in FIGS. 2A-2C, 3A-3C, and 4, and Tables 1 and 2. Local recurrence (IBTR) was investigated at 10 years since breast conserving surgery. FIG. 4 depicts the effect of RT in the combined low and moderate PDGFRb score group compared to the high PDGFRb score group. FIG. 5 depicts the correlation between PDGFRb score and clinicopathological parameters. The tables below FIGS. 2A-2C, 3A-3C, and 4 indicate the number of patients at risk. P-values are based on the cumulative incidence function (CIF) numbers over ten years since breast conserving surgery.

Table 1. RT predictive performance of PDGFRb score and interaction between PDGFRb score and RT treatment in Cox regression analysis.

TABLE 1
RT predictive performance of PDGFRb score and interaction between PDGFRb score
and RT treatment in Cox regression analysis.

| | | | Multivariable |
|---|---|---|---|
| | | | RT vs. non-RT |
| | | | incl. histological grade |
| | PDGFR | RT vs. non-RT | and age group |
| Endpoint | score group | HR (95% CI); p-value | HR (95% CI); p-value |
| IBTR, 10 years | Low | 0.25 (0.11-0.56); p = 0.001 | 0.27 (0.12-0.62); p = 0.002 |
| | Medium | 0.25 (0.13-0.48); p < 0.001 | 0.30 (0.16-0.58); p < 0.001 |
| | High | 0.61 (0.35-1.05); p = 0.073 | 0.62 (0.36-1.08); p = 0.091 |
| | Interaction | | P = 0.138 |
| | PDGFRb | | |
| | score: RT | | |
| Any recurrence, | Low | 0.50 (0.28-0.89); p = 0.018 | 0.55 (0.30-0.98); p = 0.044 |
| 10 years | Medium | 0.37 (0.23-0.60); p < 0.001 | 0.45 (0.28-0.73); p = 0.001 |
| | High | 0.70 (0.46-1.06); p = 0.089 | 0.73 (0.48-1.12); p = 0.150 |
| | Interaction | | P = 0.332 |
| | PDGFRb | | |
| | score: RT | | |
| BCSD, 15 years | Low | 1.05 (0.51-2.18); p = 0.888 | 1.17 (0.56-2.42); p = 0.676 |
| | Medium | 0.54 (0.29-1.00); p = 0.051 | 0.67 (0.35-1.26); p = 0.212 |
| | High | 0.77 (0.45-1.32); p = 0.338 | 0.82 (0.48-1.41); p = 0.470 |
| | Interaction | | P = 0.520 |
| | PDGFRb | | |
| | score: RT | | |

Table 2. Prognostic performance of PDGFRb score group in uni- and multivariable Cox regression analysis.

TABLE 2
Prognostic performance of PDGFRb score group in uni- and
multivariable Cox regression analysis.

| | | | Multivariable |
|---|---|---|---|
| | PDGFR | | incl. RT, histological |
| | score | Univariable | grade, age group |
| Endpoint | group | HR (95% CI); p-value | HR (95% CI); p-value |
| IBTR, | Low | 1 | 1 |
| 10 years | Medium | 1.51 (0.99-2.30); 0.057 | 1.43 (0.92-2.22); 0.108 |
| | High | 1.33 (0.87-2.02); 0.187 | 1.19 (0.77-1.85); 0.427 |
| Any | Low | 1 | 1 |
| recurrence, | Medium | 1.58 (1.11-2.23); 0.011 | 1.47 (1.03-2.11); 0.034 |
| 10 years | High | 1.49 (1.06-2.10); 0.021 | 1.32 (0.93-1.88); 0.125 |
| BCSD, | Low | 1 | 1 |
| 15 years | Medium | 1.37 (0.85-2.21); 0.191 | 1.37 (0.85-2.21); 0.198 |
| | High | 1.52 (0.96-2.38); 0.075 | 1.42 (0.89-2.24); 0.139 |

### Marker Evaluation

Out of 1004 cases included in the TMA, 989 cases were successfully scored (FIGS. 1, 6). Using Cohen's kappa statistics, the inter-rater agreement was in the moderate range for scoring of the average staining intensity (K=0.59) and of the positive stroma fraction (K=

Correlation with Clinicopathological Patient Characteristics

The distribution of clinicopathological variables can be seen in Table 3 (FIG. 7). A high PDGFRb score was associated with ER negativity (Spearman's p=0.098, p=0.003), young age (p=0.195, p<0.001), subtype (p=0.142, p<0.001) and a lower overall stroma fraction (p=0.064, p=0.043) in Spearman's Rank tests (FIG. 5).

### Prognostic Potential of Stromal PDGFRb Expression

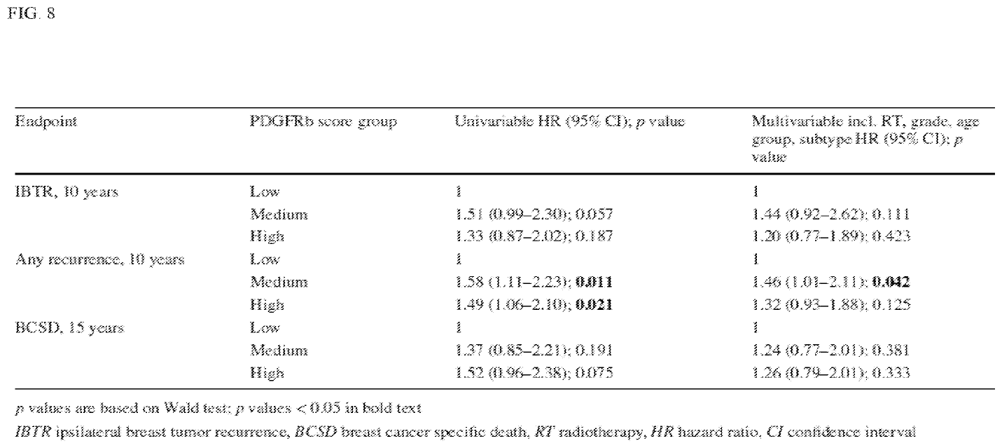

No prognostic impact was observed for any of the PDGFRb score groups with regards to IBTR at 10 years after BCS (FIG. 2A, Table 4 in FIG. 8). For any recurrence, a significantly increased risk was detected in univariable analysis for patients with a medium (HR 1.58, CI 95% 1.11-2.23, p=0.011) or high PDGFRb score (HR 1.49, CI 95% 1.06-2.10, p=0.021) as compared to the PDGFRb low score group (FIG. 2B, Table 4 in FIG. 8). In a multivariable analysis including histological grade, age, RT and subtype, the significance remained for the PDGFRb medium (HR 1.46, CI 95% 1.01-2.11, p=0.042) but not the PDGFRb high score group (HR 1.32, CI 95% 0.93-1.88, p=0.125) (Table 4 in FIG. 8). PDGFRb score was not significantly associated with risk of BCSD within 15 years from diagnosis (FIG. 2C, Table 4 in FIG. 8).

### RT-Predictive Potential of Stromal PDGFRb Expression

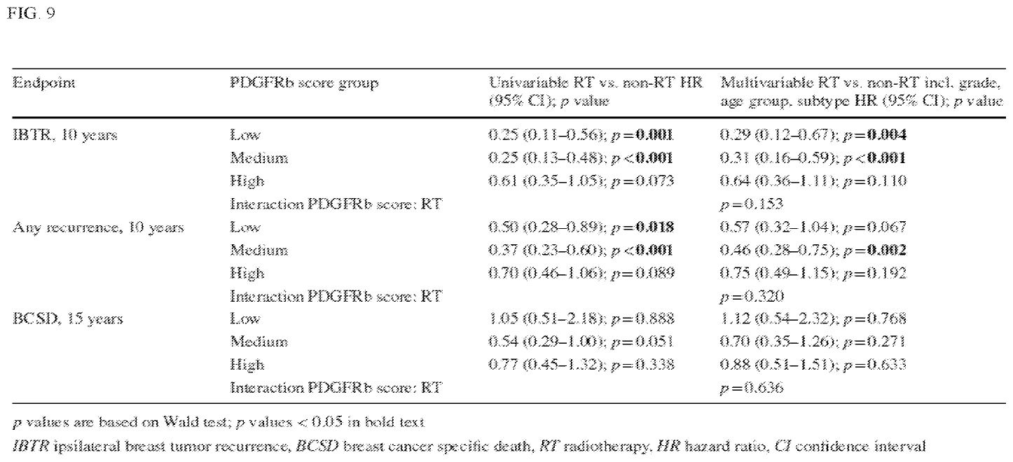

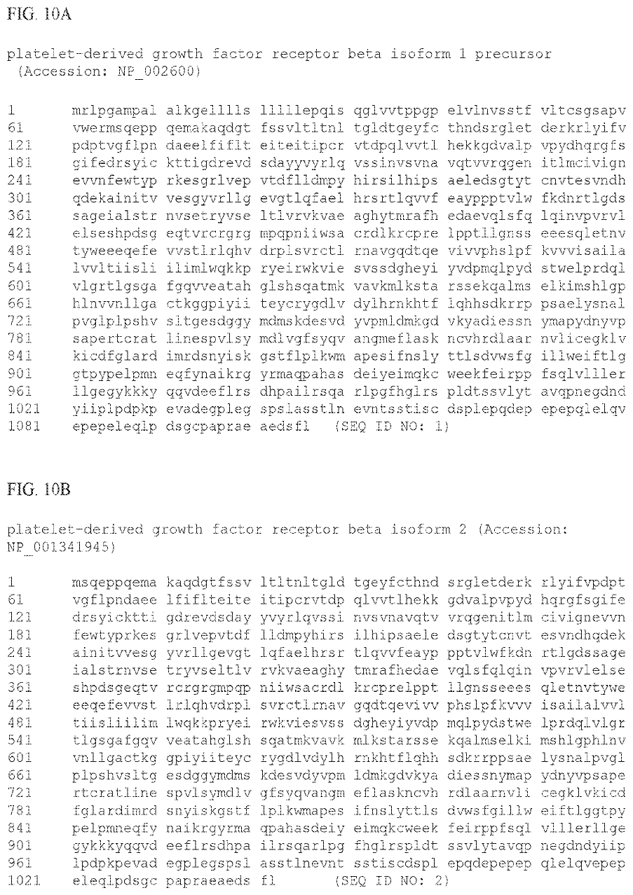

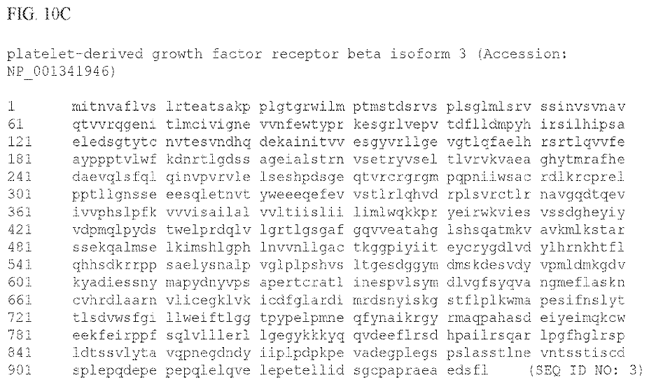

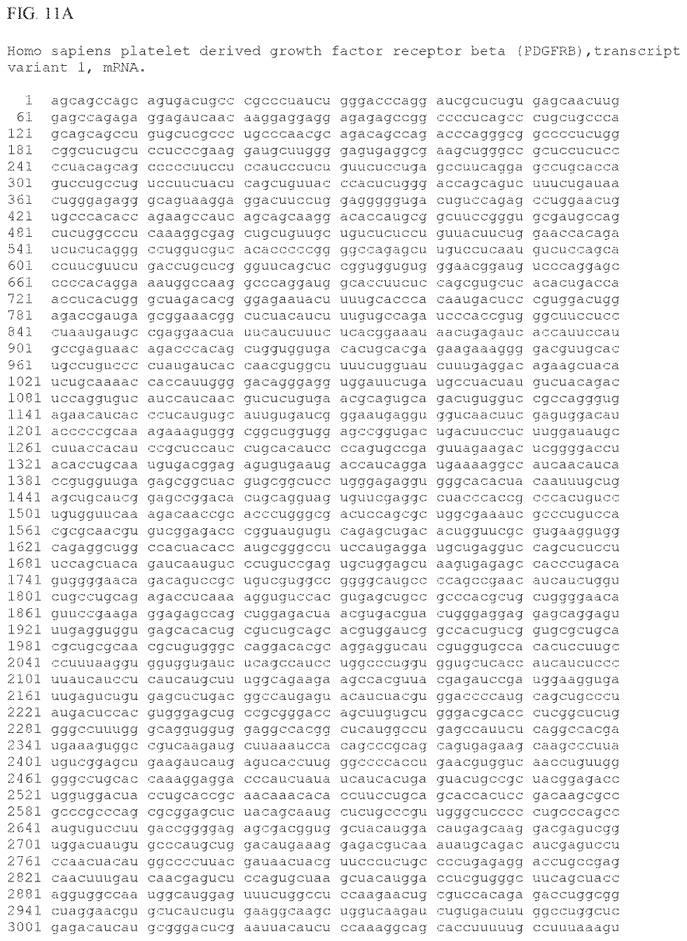

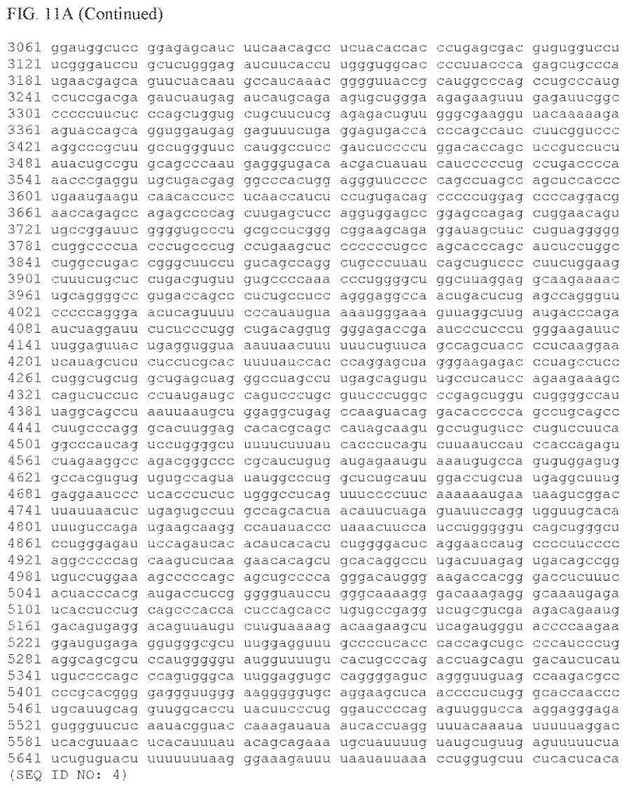

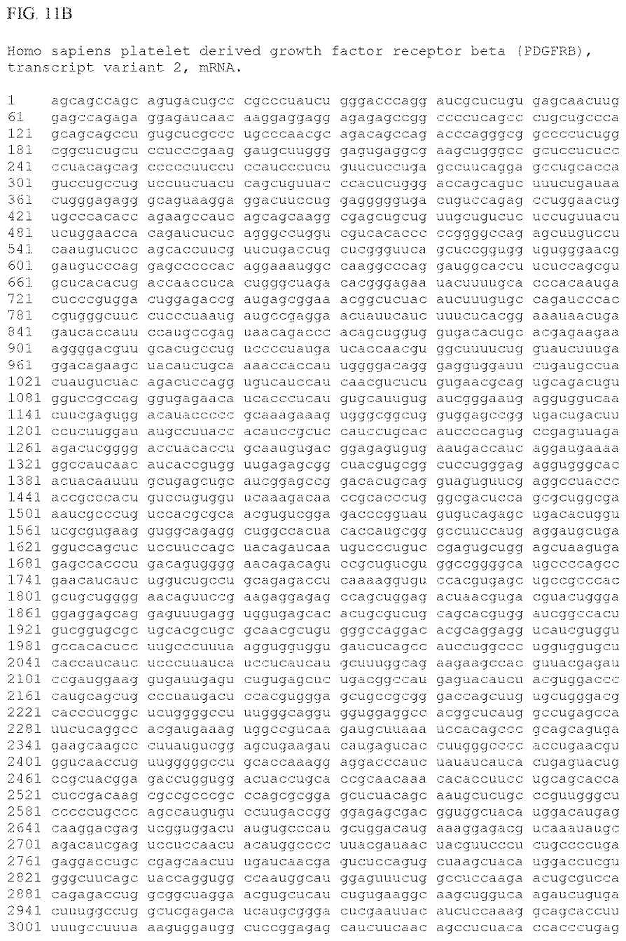

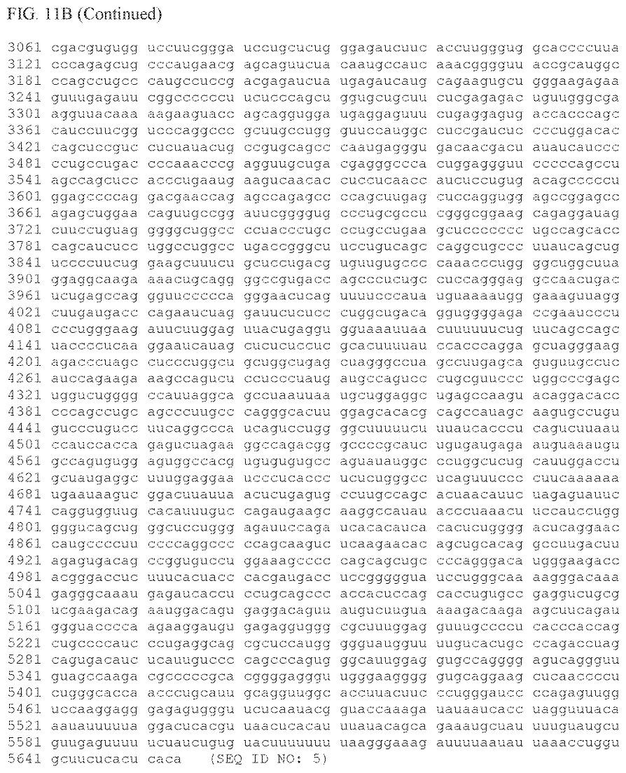

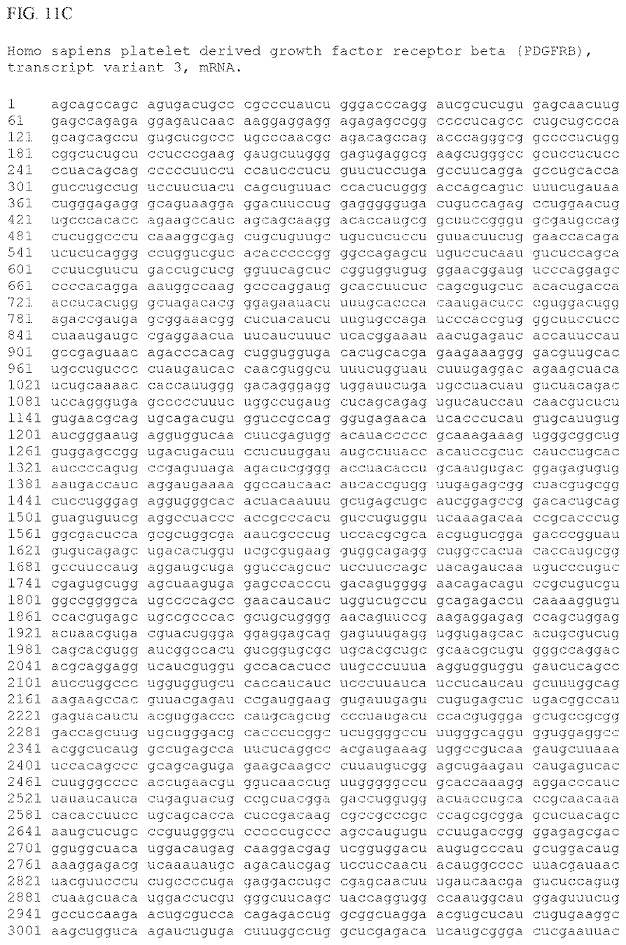

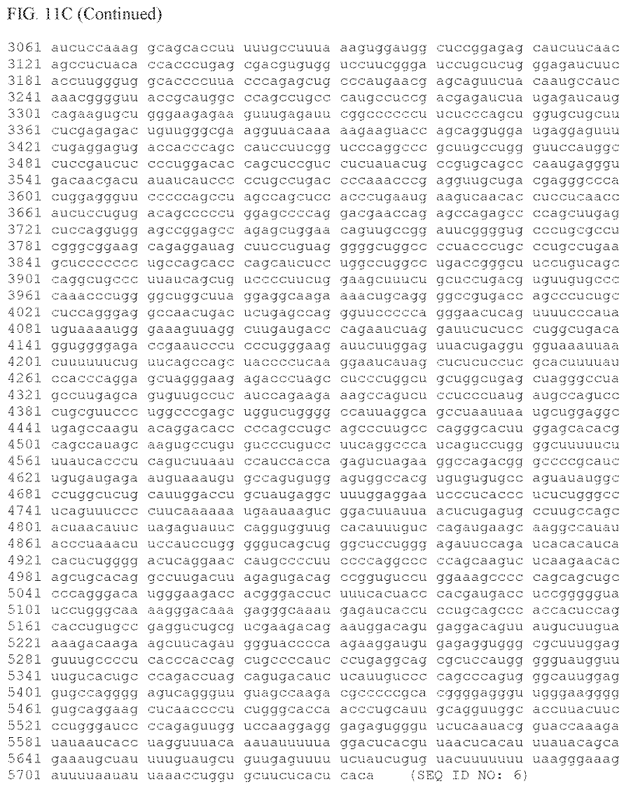

The benefit of RT regarding the risk of IBTR was significant in univariable as well as multivariable analysis including histological grade, age and subtype for the PDGFRb low [univariable: HR 0.25, CI 95% 0.11-0.56, p<0.001; multivariable: 0.29 (0.12-0.67), p=0.004] and medium [univariable: HR 0.25, CI 95% 0.13-0.48, p<0.001; multivariable: 0.31 (0.16-0.59), p<0.001] score groups but not in the PDGFRb high [univariable: HR 0.61, CI 95% 0.35-1.05, p=0.073; multivariable: 0.64 (0.36-1.11), p=0.110] score group at 10 years after BCS (FIG. 4, top panels, Table 5 in FIG. 9).

Likewise, the RT benefit regarding the risk for any recurrence was less pronounced in the PDGFRb high score group [univariable: HR 0.70, CI 95% 0.46-1.06, p=multivariable: 0.75 (0.49-1.15), p=0.192] as compared to the PDGFRb low [univariable: HR 0.50, CI 95% 0.28-0.89, p=0.018; multivariable: 0.57 (0.32-1.04), p=0.067] and medium [univariable: HR 0.37, CI 95% 0.23-0.60, p<0.001; multivariable: 0.46 (0.28-0.75), p=0.002] score groups.

No significant interaction between RT and PDGFRb score could however be detected for IBTR (p=0.153) or any recurrence (p=0.320) (FIG. 4, middle panels, Table 5 in FIG. 9). No benefit from RT regarding BCSD was observed for any of the PDGFRb score groups at 15 years after breast conserving surgery and no significant interaction between PDGFRb score and RT was noted for BCSD (p=0.636) (FIG. 4, bottom panels, Table 5 in FIG. 9).

These data suggest that patients with higher expression of PDGFRb might have an increased risk of any breast cancer recurrence, but due to correlation with younger age and ER negativity, a function of PDGFRb as independent prognostic marker could not be demonstrated. Furthermore, our analyses demonstrated, both univariably as well as multivariably, that patients of the high PDGFRb score group derive less benefit from adjuvant RT in terms of IBTR as compared to the low and medium score groups. However, the interaction test between PDGFRb and RT was not significant.

PDGFRb can be correlated with unfavorable clinicopathological variables such as ER negativity, younger age and higher histological grade. These associations were confirmed in this study, and could explain part of the prognostic effect of PDGFRb expression. However, the prognostic influence remained significant in multivariable analysis regarding any recurrence for the PDGFRb medium score group patients, which indicates that PDGFRb can provide independent prognostic information. In the present study, a tendency towards higher IBTR risk among patients with higher PDGFRb expression was also noted, although these results were not significant.

The medium and high PDGFRb groups showed an increased propensity for any recurrence in univariable analysis, while no significant differences in rate of IBTR only were observed between the groups.

The overall stroma fraction was highest among Luminal A tumors and lowest among triple negative tumors. PDGFRb score showed the opposite distribution among subtypes and was instead correlated with unfavorable clinicopathological variables.

The presented study included a large patient number and randomized design of the cohort, allowing investigation of prognostic and predictive effects differentially. The results suggest that higher stromal PDGFRb expression is associated with an increased risk of any recurrence.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 markers refers to groups having 1, 2, or 3 markers. Similarly, a group having 1-5 markers refers to groups having 1, 2, 3, 4, or 5 markers, and so forth.

All patent filings, websites, other publications, accession numbers and the like cited above or below are incorporated by reference in their entirety for all purposes to the same extent as if each individual item were specifically and individually indicated to be so incorporated by reference. Any feature, step, element, embodiment, or aspect disclosed herein can be used in combination with any other unless specifically indicated otherwise.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for treating a subject for local recurrence of invasive breast cancer, said method comprising:
providing a local cancer tissue sample from a subject who has invasive breast cancer;
analyzing the local cancer tissue sample for a level of stromal and/or epithelial PDGFRb;
treating the subject with radiotherapy if the local cancer tissue sample has a low level of PDGFRb; and
treating the subject with an alternative to standard radiotherapy if the local cancer tissue sample has a high level of PDGFRb.

2. A method of treating a subject, the method comprising:
identifying a subject with invasive breast cancer that has a high level of PDGFRb; and
administering an aggressive breast cancer therapy to the subject locally to where there is the high level of PDGFRb, wherein the aggressive breast cancer therapy is at least more than standard radiation.

3. A method of identifying a subject who will not be adequately responsive to radiation therapy, the method comprising:
identifying a subject with invasive breast cancer; and
determining if a local cancer tissue sample from the subject has a high level of PDGFRb,
wherein if the local cancer tissue sample from the subject has a high level of PDGFRb, administering an aggressive therapy to the subject,
wherein the aggressive therapy is not standard radiation therapy, and
wherein the aggressive therapy is at least: a) standard radiation with the addition of mastectomy or chemotherapy, or b) mastectomy or c) mastectomy and chemotherapy.

4. A method for recommending a treatment to a subject, said method comprising:
analyzing a local cancer tissue sample for a level of PDGFRb from a subject;
recommending that one treats the subject with standard radiotherapy if the local cancer tissue sample has a low level of PDGFRb; and
recommending that one treats the subject with an alternative to standard radiotherapy if the local cancer tissue sample has a high level of PDGFRb.

5. A method for preventing an invasive breast cancer recurrence in a subject, the method comprising:
providing a cancer tissue sample from a subject who has invasive breast cancer;
analyzing the cancer tissue sample for a level of PDGFRb;
administering standard radiotherapy if the local cancer tissue sample has a low level of PDGFRb; and
administering an alternative to standard radiotherapy if the local cancer tissue sample has a high level of PDGFRb.

6. A method for preventing a local and/or regional recurrence of an invasive breast cancer in a subject, the method comprising:
receiving standard radiotherapy if a local cancer has a low level of PDGFRb; or
receiving an alternative to standard radiotherapy if the invasive breast cancer has a high level of PDGFRb.

7. A method of modifying a treatment for a subject, the method comprising:
identifying a subject with invasive breast cancer that has a high level of PDGFRb; and
administering a breast cancer therapy to the subject, wherein the breast cancer therapy is more aggressive than a traditional breast cancer therapy, wherein the traditional breast cancer therapy is one recommended for the subject, based on the subject's risk factors excluding PDGFRb levels.

8. The method of claim 7, wherein the traditional breast cancer therapy is defined by the NCCN guidelines as of October 2020.

9. The method of any one of the preceding claims, wherein low level of PDGFRb denotes a level of protein expression.

10. The method of any one of the preceding claims, wherein low level of PDGFRb denotes a level of mRNA present in the sample.

11. The method of any one of the preceding claims, wherein high level of PDGFRb denotes a level of protein expression.

12. The method of any one of the preceding claims, wherein a high or low level of PDGFRb is determined as a value relative to a control.

13. The method of claim 12, wherein the control comprises a positive, a negative, or a positive and a negative control.

14. The method of claim 12 or 13, wherein the control is an internal control or an external control or both.

15. The method of claim 12 or 13, wherein the control includes a level defined to one or more housekeeping genes.

16. The method of any one of the preceding claims, wherein high or low levels of PDGFRb are defined by a comparison of PDGFRb levels from local tissue sample to a control sample in a healthy subject.

17. The method of any one of the preceding claims, wherein high or low levels of PDGFRb are defined by a comparison of PDGFRb levels from the local tissue sample to a control sample from a tissue in the subject that does not include invasive cancer.

18. The method of any one of the preceding claims, wherein high or low level of PDGFRb are defined by a comparison to a standardized level set by a level of expression of a house keeping gene.

19. The method of any one of the preceding claims, wherein treating the subject with standard radiotherapy denotes a therapy in line with the guidelines in the NCCN guidelines.

20. The method of claim 19, wherein the NCCN guidelines are as of 2020.

21. The method of any one of the preceding claims, wherein treating the subject with an alternative to standard radiotherapy denotes either a) administering a more intense level of therapy than that outlined in the NCCN guidelines, orb) radiation boost with higher dose levels or with broader indications than in current NCCN guidelines, mastectomy, concurrent radiochemotherapy.

22. The method of any one of the preceding claims, wherein treating the subject with an alternative to standard radiotherapy denotes applying radiotherapy at an intensity of more than: 25 fractions of 2 Gy each (total 50 Gy), 15 fractions of 2.67 Gy each (total 40 Gy), 16 fractions of 2.66 Gy each (total 42.5 Gy) or 5 fractions of 5.2 Gy each (total 26 Gy).

23. The method of any one of the preceding claims, wherein treating the subject with an alternative to standard radiotherapy denotes applying radiotherapy in combination with at least one of the following: chemotherapy, endocrine therapy, anti-HER2 therapy, immunotherapy, PARP inhibitor therapy, or other targeted therapies such as tyrosine kinase inhibitors or monoclonal antibodies against PDGFRb.

24. The method of any one of the preceding claims, wherein high PDGFRb denotes the subject who has PDGFRb levels at the highest quartile of a population of PDGFRb levels of a population of people from the SweBCG91RT clinical trial of 1991-97.

25. The method of any one of the preceding claims, wherein a high or low PDGFRb level is determined by a combination of a) intensity of staining and b) percent of positive fraction of the tumor stained, wherein greater amounts in either a) orb) result in an increased PDGFRb level.

26. The method of claim 25, wherein a) is defined into four parts, and wherein b) is defined into five parts.

27. The method of any one of the preceding claims, wherein high PDGFRb denotes the subject has PDGFRb levels at the highest 10% of a population of PDGFRb levels of a population of people.

28. The method of any one of the preceding claims, wherein high or low PDGFRb is determined as a function of staining of the fraction of whole stroma that is positive and a level of expression.

29. The method of claim 28, wherein fraction of staining is multiplied by the level of expression.

30. The method of any one of the preceding claims, wherein a level of PDGFRb is analyzed as a continuous metric so that a continuous risk assessment is further provided to the subject.

31. The method of any one of the preceding claims, wherein high or low PDGFRb is determined by measuring the stromal PDGFRb staining intensity in the area of the tumor-associated stroma displaying highest PDGFRb expression.

32. The method of any one of the preceding claims, wherein the local cancer tissue sample is analyzed for a level of stromal PDGFRb.

33. The method of any one of the preceding claims, wherein the local cancer tissue sample is analyzed for a level of epithelial PDGFRb.

34. A method of treating a subject, the method comprising:
identifying an incremental risk to a subject of a local recurrence of an invasive breast cancer based on a level of PDGFRb in a sample of an invasive breast cancer in the subject; and
administering an aggressive breast cancer therapy to the subject based upon the incremental risk, wherein a higher incremental risk will increase:
a) a likelihood of an aggressive breast cancer therapy that is at least more than what would be recommended by the NCCN;
b) the aggressiveness of the aggressive breast cancer; or
c) both a) and b).
